(19) United States
(12) Patent Application Publication
(10) Pub. No.: US 2009/0081137 A1
(43) Pub. Date: Mar. 26, 2009

(54) Crosslinked Polytetrahydrofuran-Containing Polyurethanes

(75) Inventors: Son Nguyen Kim, Hemsbach (DE); Thomas Kaiser, Hassloch (DE); Ralf Hauck, Maxdorf (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/658,268

(22) PCT Filed: Jul. 22, 2005
(86) PCT No.: PCT/EP05/08040

§ 371 Date: Jan. 24, 2007

(30) Foreign Application Priority Data
Jul. 26, 2004 (DE) 10 2004 036 146.0

(51) Int. Cl. A61K 8/87; C08G 18/10; A61K 8/898; A61Q 17/04; A61Q 19/00; A61Q 5/12; A61Q 3/02; A61Q 19/10; A61K 31/785; C08G 77/04

(52) U.S. Cl. 424/59; 528/28; 528/59; 424/78.03; 424/70.11; 424/70.12; 424/61

(57) ABSTRACT

The invention relates to a cross-linked polyurethane, which contains, in an integrated manner, at least one polytetrahydrofuran and one polyisocyanate mixture, to cosmetic or pharmaceutical agents containing a polyurethane of the aforementioned type, and to the use of these polyurethanes.

The present invention relates to a crosslinked polyurethane which comprises at least one polytetrahydrofuran and a polyisocyanate mixture, to cosmetic or pharmaceutical compositions which comprise such a polyurethane, and to the use of these polyurethanes.

Cosmetically and pharmaceutically acceptable water-soluble or water-dispersible polymers are used widely in cosmetics and medicine. They are used, for example, quite generally as thickeners for diverse types of formulations, such as, for example, gels, creams or emulsions. For these applications, use is often made of branched or crosslinked water-soluble polymers with anionic functionalities, such as, for example, crosslinked polyacrylic acid. For hair cosmetics in particular, crosslinked polymers with film-forming properties are used as conditioners in order to improve the dry and wet combability, the feel to the touch, the shine and/or the appearance of the hair, and also to impart antistatic properties to the hair. Besides the abovementioned carboxylate-group-containing polymers, the conditioners used are often crosslinked polymers with cationic functionalities which have a high affinity to the surface of the hair, which is negatively charged as the result of its structure. These include, for example, crosslinked copolymers of N-vinylpyrrolidone, quaternized N-vinylimidazole, acrylamide and diallyldimethylammonium chlorid (DADMAC).

The provision of products with a complex profile of properties often presents difficulties. For example, there is a need for polymers for cosmetic and pharmaceutical compositions which firstly have good film-forming properties and secondly have good flexibility of the resulting films.

WO 94/03510 describes the use of polyurethanes of
a) at least one compound which contains two or more active hydrogen atoms per molecule,
b) at least one acid- or salt-group-containing diol and
c) at least one diisocyanate
   with a glass transition temperature of at least 15° C. and acid numbers of from 12 to 150 or the salts of these polyurethanes in cosmetic preparations and as binders or coatings in pharmaceutical preparations.

EP-A-619 111 describes the use of polyurethanes based on organic diisocyanates, diols and 2,2-hydroxymethyl-substituted carboxylates of the formula

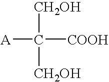

where A is a hydrogen atom or a C₁-C₂₀-alkyl group, in hair-setting compositions. At least some of the carboxylic acid groups here are neutralized with an organic or inorganic base. The diols here have a molecular weight in the range from 300 to 20 000, with polytetrahydrofurans also been specified, inter alia, as suitable diol component.

WO 94/13724 describes the use of cationic polyurethanes and polyureas of
(a) at least one diisocyanate, which can already be reacted beforehand with one or more compounds which comprise two or more active hydrogen atoms per molecule, and
(b) at least one diol comprising one or more tertiary; quaternary or protonated tertiary amine nitrogen atoms, primary or secondary amino alcohol, primary or secondary diamine or primary or secondary triamine
   with a glass transition temperature of at least 25° C. and an amine number of from 50 to 200, based on the nonquaternized or protonated compounds, or other salts of these polyurethanes and polyureas as auxiliaries in cosmetic and pharmaceutical preparations.

WO 01/16200 describes a cosmetic composition comprising water-soluble or water-dispersible polyurethanes of an oligomer or polymer of
A) at least one diisocyanate,
B) at least one compound with at least two groups reactive toward isocyanate groups which is chosen from
   B1) aliphatic and cycloaliphatic polyols, polyamines and/or amino alcohols,
   B2) polyetherols and/or diaminopolyethers,
   B3) polysiloxanes with at least two active hydrogen atoms per molecule,
   B4) polyesterpolyols,
   and mixtures thereof, and
C) if appropriate at least one dicarboxylic acid and/or hydroxycarboxylic acid,
   where the oligomer comprises, per molecule, at least two urethane and/or urea groups and additionally at least two further functional groups which are chosen from hydroxyl groups, and primary and/or secondary amino groups.

EP-A-938 889 describes a cosmetic composition comprising at least one water-soluble or water-dispersible polyurethane of
a) at least one polymer with two active hydrogen atoms per molecule which is chosen from polytetrahydrofurans, polysiloxanes and mixtures thereof,
b) at least one polyesterdiol,
c) at least one compound with a molecular weight in the range from 56 to 300 which comprises two active hydrogen atoms per molecule,
d) at least one compound which has two active hydrogen atoms and at least one anionogenic and/or anionic group per molecule,
e) at least one diisocyanate,
   or the salts thereof, where the polyurethane comprises no unit originating from a primary or secondary amine which has an ionogenic and/or ionic group.

WO 99/58100 describes a cosmetic composition comprising at least one crosslinked, water-soluble or water-dispersible polyurethane of at least one polyurethane prepolymer with terminal isocyanate groups and at least one polymer with groups reactive toward isocyanate groups, where at least one of the components comprises a siloxane group.

WO 01/85821 describes polyurethanes based on at least one polyether with two active hydrogen atoms per molecule and their use for modifying rheological properties.

DE-A-102 59 036 describes allyl-group-containing polyether urethanes, polymers which comprise these in copolymerized form, and cosmetic or pharmaceutical compositions based on these polymers.

EP-A-957 119 describes crosslinked, water-soluble or water-dispersible polyurethanes of
A) at least one water-soluble or water-dispersible polyurethane prepolymer with terminal isocyanate groups of
   a) at least one compound with a molecular weight in the range from 56 to 300 which comprises two active hydrogen atoms per molecule,
   b) at least one polymer with two active hydrogen atoms per molecule,
   c) at least one compound which has two active hydrogen atoms and at least one ionogenic and/or ionic group per molecule,
   d) at least one diisocyanate,
B) at least one polymer with groups reactive toward isocyanate groups which are chosen from hydroxyl groups and primary and secondary amino and/or carboxyl groups,
   or the salts thereof, and the use of these polyurethanes as auxiliaries in cosmetics.

WO 03/085019 describes crosslinked polyurethanes based on polytetrahydrofuran and the use thereof in cosmetic and pharmaceutical compositions.

The object forming the basis of the present invention is to provide novel film-forming polymers which are suitable for the preparation of cosmetic and/or pharmaceutical compositions. Besides an overall good application profile for this area of use, these polymers should primarily have very good film-forming properties which leads to films with high setting effect and at the same time high flexibility. In addition, the resulting films should be as clear as possible.

Surprisingly, we have now found that this object is achieved by crosslinked polyurethanes which comprise, in incorporated form, at least one polytetrahydrofuran and, as isocyanate component, a polyisocyanate mixture which comprises isophorone diisocyanate and hexamethylene diisocyanate. The invention therefore provides a crosslinked polyurethane which comprises, in incorporated form,
A) at least one polytetrahydrofuran with two terminal hydroxyl groups per molecule and a number-average molecular weight in the range from 650 to 2000,
B) at least one compound which comprises more than two active hydrogen atoms per molecule,
C) at least one compound which comprises at least two active hydrogen atoms and at least one anionogenic and/or anionic group per molecule,
D) a polyisocyanate mixture which comprises isophorone diisocyanate and hexamethylene diisocyanate, and
E) at least one compound different from A) to D) with at least two active hydrogen atoms and a molecular weight of from 60 to 5000,
   and the salts thereof.

A suitable embodiment of the crosslinked polyurethanes according to the invention are those which additionally comprise, in incorporated form, as component F) at least one polysiloxane with at least two active hydrogen atoms per molecule.

For the purposes of the present invention, the expression alkyl comprises straight-chain and branched alkyl groups. Suitable short-chain alkyl groups are, for example, straight-chain or branched C₁-C₈-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc. Suitable longer-chain C₈-C₃₀-alkyl and C₈-C₃₀-alkenyl groups are straight-chain and branched alkyl and alkenyl groups. Preferably, these are predominantly linear alkyl radicals, as also arise in natural or synthetic fatty acids and fatty alcohols, and oxo alcohols which may if appropriate be additionally mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular is phenyl, tolyl, xylyl or mesityl.

In the text below, compounds which can be derived from acrylic acid and methacrylic acid are sometimes referred in shortened form by adding the syllable “(meth)” to the compound derived from acrylic acid.

Polymers based on the crosslinked polyurethanes according to the invention can advantageously be formulated as gels under standard conditions (20° C.). “Gel-like consistency” is shown by compositions which have a higher viscosity than a liquid and which are self-supporting, i.e. they retain a shape given to them without shape-stabilizing coating. In contrast to solid formulations, however, gel-like formulations can be readily deformed under the application of shear forces. The viscosity of the gel-like compositions is preferably in a range of greater than 600 to about 60 000 mPas. The gels are preferably hair gels which have a viscosity of from preferably 6000 to 30 000 mPas.

For the purposes of the present invention, water-soluble monomers and polymers are understood as meaning monomers and polymers which dissolve in water in an amount of at least 1 g/l at 20° C. Hydrophilic monomers are water-soluble or at least water-dispersible. Water-dispersible polymers are understood as meaning polymers which disintegrate into dispersible particles under the application of shear forces, for example by stirring. The crosslinked polyurethanes according to the invention are preferably water-soluble or water-dispersible. The water-dispersible polyurethanes according to the invention are generally suitable for the preparation of microdispersions.

### Component A)

Component A) is preferably chosen from polytetrahydrofurans of the general formula

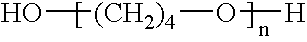

where n=4 to 40, preferably 6 to 35,
and mixtures thereof.

These polytetrahydrofurans have a number-average molecular weight in the range from 650 to 2000, preferably 750 to 1800, in particular 800 to 1500.

Suitable polytetrahydrofurans can be prepared by cationic polymerization of tetrahydrofuran in the presence of acidic catalysts, such as, for example, sulfuric acid or fluorosulfuric acid. Such preparation processes are known to the person skilled in the art.

### Component B)

Component B) is compounds which comprise more than two active hydrogen atoms per molecule. Suitable as component B) are compounds which have more than two groups reactive toward NCO groups, which are preferably chosen from hydroxyl groups, and primary and secondary amine groups.

Preferably, the compounds of component B) are chosen from triols, polyols with more than three hydroxyl groups, triamines, polyamines with more than three primary or secondary amino groups, amine- and/or hydroxyl-group-containing polymers and mixtures thereof.

Preferably suitable as component B) are compounds with a molecular weight in the range from about 80 to less than 1000 g/mol and with about 3 to 20, particularly preferably 3 to 10, in particular 3 to 5, groups reactive toward NCO groups.

Also preferably suitable as component B) are polymers with a number-average molecular weight of at least 1000 and which have a hydroxyl number of at least 0.1 g of KOH/g and/or an amine number of at least 0.1 g of KOH/g. These include preferably hydroxyl-group-containing polymers which have an OH number in the range from 0.3 to 60, particularly preferably from 0.9 to 30 and in particular from 1.5 to 21. These also include amine-group-containing polymers which have an amine number in the range from 0.3 to 60, particularly preferably from 0.9 to 30 and in particular from 1.5 to 21. These also include hydroxyl- and amine-group-containing polymers in which the sum of hydroxyl and amine number is in a range from 0.3 to 60, preferably from 0.9 and 30 and in particular from 1.5 to 21.

The compounds of component B) are preferably chosen from triols and polyols of higher functionality having 3 to 100, preferably 3 to 70, carbon atoms. Preferred triols are, for example, glycerol and trimethylolpropane. Preferred triols B) are also the triesters of hydroxycarboxylic acids with trihydric alcohols. These are preferably triglycerides of hydroxycarboxylic acids, such as, for example, lactic acid, hydroxystearic acid and ricinoleic acid. Also suitable are naturally occurring mixtures which comprise hydroxycarboxylic acid triglycerides, in particular castor oil. Preferred polyols B) of higher functionality are, for example, erythritol, pentaerythritol and sorbitol.

Preferred triamines B) are, for example, diethylenetriamine, N,N′-diethyldiethylenetriamine etc. Preferred polyamines of higher functionality are, for example, triethylenetetramine etc. and α,ω-diaminopolyethers which can be prepared by amination of polyalkylene oxides with ammonia.

Further preferred compounds of component B) are hydroxyl- and/or amine-group-containing polymers which have a number-average molecular weight of from about 1000 to 200 000, preferably about 5000 to 100 000. These are particularly preferably amine-group-containing polymers.

In a preferred embodiment, component B) is an amine- and/or hydroxyl-group-containing polymer which is obtainable by free-radical polymerization of α,β-ethylenically unsaturated monomers. Here, at least one α,β-ethylenically unsaturated monomer is used which has at least one functional group which is chosen from hydroxyl groups, primary or secondary amine groups or groups which can be converted to such amine groups. This monomer is preferably chosen from esters of α,β-ethylenically unsaturated mono- and dicarboxylic acids with amino alcohols which have at least one primary or secondary amino group, amides of α,β-ethylenically unsaturated mono- and dicarboxylic acids with diamines which have at least one primary or secondary amino group, esters of α,β-ethylenically unsaturated mono- and dicarboxylic acids with diols, amides of α,β-ethylenically unsaturated mono- and dicarboxylic acids with amino alcohols which have a primary or secondary amino group, N,N-diallylamine and mixtures thereof (=monomer a)).

The amine-group-containing polymers preferably comprise 0.1 to 20% by weight, particularly preferably 0.3 to 10% by weight, in particular 0.5 to 7% by weight, especially at most 4% by weight, based on the total weight of the monomers used for the polymerization, of at least one monomer a) in copolymerized form.

Suitable compounds a) are, for example, the esters of α,β-ethylenically unsaturated mono- and dicarboxylic acids with amino alcohols, preferably C₂-C₁₂-amino alcohols. These may preferably be C₁-C₈-monoalkylated on the amine nitrogen. Suitable acid components of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof. Preference is given to N-methylaminoethyl (meth)acrylate, N-ethylaminoethyl (meth)acrylate, N-(n-propyl)aminoethyl (meth)acrylate, N-(n-butyl)aminoethyl (meth)acrylate, tert-butylaminoethyl (meth)acrylate. Particular preference is given to N-tert-butylaminoethyl methacrylate.

Suitable monomers a) are also the amides of the abovementioned α,β-ethylenically unsaturated mono- and dicarboxylic acids with diamines which have at least one primary or secondary amino group.

Suitable monomers a) are, for example, N-methylaminoethyl(meth)acrylamide, N-ethylaminoethyl(meth)acrylamide, N-(n-propyl)aminoethyl(meth)acrylamide, N-(n-butyl)aminoethyl(meth)acrylamide and N-tert-butylaminoethyl(meth)acrylamide.

Suitable monomers a) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Suitable monomers a) are also 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

A preferred monomer a) is also N,N-diallylamine.

Preferred monomers a) are tert-butylaminoethyl methacrylate, diallylamine and mixtures thereof.

The amine-group- and/or hydroxyl-group-containing polymers can additionally comprise at least one further nonionic amide-group-containing α,β-ethylenically unsaturated monomer different from the abovementioned monomers a) and copolymerizable therewith in copolymerized form (=monomer b)). This is preferably a water-soluble monomer.

The amine-group-containing polymers preferably comprise 0 to 80% by weight, particularly preferably 1 to 75% by weight, in particular 10 to 70% by weight, based on the total weight of the monomers used for the polymerization, of at least one monomer b) in copolymerized form.

This component b) is preferably chosen from N-vinyllactams, N-vinylamides of saturated C₁-C₈-monocarboxylic acids, primary amides of α,β-ethylenically unsaturated monocarboxylic acids and their N-alkyl and N,N-dialkyl derivatives, and mixtures thereof.

Preferably, the amine-group-containing polymers additionally comprise at least one N-vinyllactam b) in copolymerized form. Suitable monomers b) are unsubstituted N-vinyllactams and N-vinyllactam derivatives, which may, for example, have one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. Preference is given to using N-vinylpyrrolidone and/or N-vinylcaprolactam.

Open-chain N-vinylamide compounds suitable as monomers b) are, for example, N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide.

Suitable monomers b) are also acrylamide and methacrylamide.

Suitable monomers b) are also N—C₁-C₈-alkyl- and N,N-di(C₁-C₈-)alkylamides of α,β-ethylenically unsaturated monocarboxylic acids. Suitable additional monomers c) are N-methyl(meth)acrylamide, N-ethyl(meth)acrylamide, N-propyl(meth)acrylamide, N-isopropyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide.

Suitable monomers b) are also N-(n-octyl)(meth)acrylamide, N-(1,1,3,3-tetramethyl-butyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide.

The amine-group-containing polymers can additionally comprise at least one, preferably water-soluble, monomer in copolymerized form which is chosen from α,β-ethylenically unsaturated compounds with anionogenic and/or anionic groups (=monomer c)). The amine-group-containing polymers preferably comprise 0 to 30% by weight. particularly preferably 0.1 to 20% by weight, in particular 0.5 to 15% by weight, based on the total weight of the monomers used for the polymerization, of at least one monomer c) in copolymerized form.

The anionogenic/anionic monomers c) are preferably chosen from monoethylenically unsaturated carboxylic acids, sulfonic acids, phosphonic acids and mixtures thereof.

The monomers c) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloracrylic acid, crotonic acid, maleic acid, maleic anhydride, itaconic acid, citraconic acid, mesaconic acid, glutaconic acid, aconitic acid and fumaric acid. The monomers c) also include the half-esters of monoethylenically unsaturated dicarboxylic acids having 4 to 10, preferably 4 to 6, carbon atoms, e.g. of maleic acid, such as monomethyl maleate. The monomers c) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxy-propylsulfonic acid, styrenesulfonic acid, 2-acrylamido-2-methylpropanesulfonic acid, vinylphosphonic acid and allylphosphonic acid. The monomers c) also include the salts of the abovementioned acids, in particular the sodium, potassium and ammonium salts, and also the salts with the abovementioned amines.

The monomers c) can be used as such or as mixtures with one another. The weight fractions given all refer to the free base form or the free acid form.

Particularly preferred monomers c) are acrylic acid, methacrylic acid and mixtures thereof.

The amino-group and/or hydroxyl-group-containing polymers can additionally comprise at least one further monomer different from the amine-group-containing monomers a) in copolymerized form, which is chosen from α,β-ethylenically unsaturated compounds with cationogenic and/or cationic groups (=monomer d)). The polymers preferably comprise 0 to 30% by weight, particularly preferably 0.1 to 20% by weight, in particular 0.5 to 15% by weight, based on the total weight of the monomers used for the polymerization, of at least one monomer d) in copolymerized form.

The cationogenic and/or cationic groups of component d) are preferably nitrogen-containing groups, such as tertiary amino groups and quaternary ammonium groups. Charged cationic groups can be produced from amine nitrogens either by protonation, e.g. with carboxylic acids, such as lactic acid, or mineral acids, such as phosphoric acid, sulfuric acid and hydrochloric acid, or by quaternization, e.g. with alkylating agents, such as C₁-C₄-alkyl halides or sulfates. Examples of such alkylating agents are ethyl chloride, ethyl bromide, methyl chloride, methyl bromide, dimethyl sulfate and diethyl sulfate.

Suitable monomers d) are, for example, N,N-dialkylaminoalkyl (meth)acrylates, such as N,N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate, N,N-dimethylaminocyclohexyl (meth)acrylate etc.

Suitable monomers d) are also N,N-dialkylaminoalkyl(meth)acrylamides, such as N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[2-(diethylamino)ethyl]methacrylamide, N-[4-(dimethylamino)cyclohexyl]acrylamide and N-[4-(dimethylamino)cyclohexyl]methacrylamide. Preference is given to N,N-dimethylaminopropyl acrylate, N,N-dimethylaminopropyl methacrylate, N-[3-(dimethylamino)propyl]acrylamide and N-[3-(dimethylamino)-propyl]methacrylamide.

Suitable monomers d) are also vinyl- and allyl-substituted nitrogen heterocycles, such as vinylimidazole, N-vinyl-2-alkylimidazoles, e.g. N-vinyl-2-methylimidazole, and 2- and 4-vinylpyridine, 2- and 4-allylpyridine, and the salts thereof.

Suitable monomers d) are also alkylallylamines and allylammonium salts, such as diallylmethylamine and diallyidimethylammonium chloride (DADMAC).

As monomer d) preference is given to N-vinylimidazole.

If, for the preparation of the amine- and/or hydroxyl-group-containing polymers, monomers with cationogenic/cationic groups which are chosen from the corresponding monomers a) and/or d), and anionogenic/anionic monomers c) are used, then these monomers may at least sometimes be used together in the form of so-called “salt pairs” for the polymerization. Preferred combinations of components a) and/or d) with c), which can be used, for example, as salt pair for the free-radical polymerization, are (meth)acrylic acid/N-tert-butylaminoethyl (meth)acrylate and (meth)acrylic acid/vinylimidazole. In general, the content of the monomers used in the form of “salt pairs” is 0 to 50% by weight, preferably 0.1 to 40% by weight, based on the total weight of the monomers used for the polymerization. The use of “salt pairs” has proven useful particularly when the amino- and/or hydroxyl-group-containing polymers comprise monomers b) in copolymerized form which are chosen from vinylpyrrolidone, vinylcaprolactam, vinylformamide and mixtures thereof.

The amine-group- and/or hydroxyl-group-containing polymers can additionally comprise at least one further monomer e) in copolymerized form which is preferably chosen from esters of α,β-ethylenically unsaturated mono- and dicarboxylic acids with C₁-C₃₀-alkanols, esters of vinyl alcohol and allyl alcohol with C₁-C₃₀-monocarboxylic acids, vinyl ethers, vinyl aromatics, vinyl halides, vinylidene halides, C₁-C₈-monoolefins, nonaromatic hydrocarbons with at least two conjugated double bonds and mixtures thereof.

Preferably, the amine-group- and/or hydroxyl-group-containing polymers comprise up to 50% by weight, particularly preferably up to 30% by weight and especially preferably up to 15% by weight, based on the total weight of the monomers used for the polymerization, of at least one monomer e) in copolymerized form. If, for the polymerization, at least one monomer e) is used, then it is preferably in an amount of at least 0.1% by weight, particularly preferably of at least 1% by weight.

Suitable monomers e) are then methyl (meth)acrylate, methyl ethacrylate, ethyl (meth)acrylate, ethyl ethacrylate, n-butyl (meth)acrylate, tert-butyl (meth)acrylate, tert-butylethacrylate, n-octyl (meth)acrylate, 1,1,3,3-tetramethylbutyl (meth)acrylate, ethylhexyl (meth)acrylate, n-nonyl (meth)acrylat, n-decyl (meth)acrylate, n-undecyl (meth)acrylate, tridecyl (meth)acrylat, myristyl (meth)acrylate, pentadecyl (meth)acrylate, palmityl (meth)acrylate, heptadecyl (meth)acrylate, nonadecyl (meth)acrylat, arrachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate and mixtures thereof. Preferred monomers e) are the esters of α,β-ethylenically unsaturated mono- and dicarboxylic acids with C₁-C₄-alkanols.

Suitable monomers e) are also vinyl acetate, vinyl propionate, vinyl butyrate and mixtures thereof.

Suitable monomers e) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The abovementioned monomers e) can be used individually or in the form of arbitrary mixtures.

Particular preference is given to amine-group-containing polymers which comprise
N-tert-butylaminoethyl methacrylate
vinylimidazole
vinylpyrrolidone and
methacrylic acid
   in copolymerized form.

Particular preference is given to amine-group-containing polymers which comprise
0.1 to 10% by weight of N-tert-butylaminoethyl methacrylate
50 to 99.9% by weight of vinylpyrrolidone,
0 to 40% by weight of methacrylic acid and vinylimidazole (molar ratio 1:1)
   in copolymerized form.

The amino-group-containing polymers are prepared by customary processes known to the person skilled in the art, e.g. by solution, precipitation, suspension or emulsion polymerization. Preference is given to preparation by solution or precipitation polymerization.

Preferred solvents for solution polymerization are aqueous solvents, such as water, water-miscible solvents and mixtures of water with water-miscible solvents, for example ketones, such as acetone and methyl ethyl ketone, alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol and the methyl or ethyl ethers of the dihydric alcohols, diethylene glycol, triethylene glycol, polyethylene glycols with number-average molecular weights up to about 3000, glycerol and dioxane.

The precipitation polymerization takes place, for example, in an ester, such as ethyl acetate or butyl acetate as solvent. The resulting polymer particles precipitate out of the reaction solution and can be isolated by customary processes, such as filtration by means of subatmospheric pressure. With precipitation polymerization, polymers with higher molecular weights are generally obtained than in the case of solution polymerization.

The polymerization temperatures are preferably in a range from about 30 to 120° C., particularly preferably 40 to 100° C. The polymerization usually takes place under atmosphere pressure, although it can also proceed under reduced or increased pressure. A suitable pressure range is between 1 and 5 bar.

To prepare the polymers, the monomers can be polymerized with the help of initiators which form free radicals.

Initiators which can be used for the free-radical polymerization are the peroxo and/or azo compounds suitable for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, 2,2′-azobis(2-amidinopropane) hydrochloride (V50 from Wako Pure Chemicals Industries, Ltd.), or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{I}.

To adjust the molecular weight, the polymerization can be carried out in the presence of at least one regulator. Regulators which may be used are the customary compounds known to the person skilled in the art, such as, for example, sulfur compounds, e.g. mercaptoethanol, 2-ethylhexyl thioglycolate, thioglycolic acid or dodecyl mercaptan, and tribromochloromethane or other compounds which have a regulating effect on the molecular weight of the resulting polymers. A preferred regulator is cysteine.

### Component C)

Preferred compounds C) with two active hydrogen atoms and at least one anionogenic and/or anionic group per molecule are, for example, compounds with carboxylate, sulfonate and/or phosphate groups. As component C), particular preference is given to 2,2-hydroxymethylalkylcarboxylic acids, such as dimethylolpropanoic acid, and mixtures which comprise 2,2-hydroxymethylalkylcarboxylic acids, such as dimethanolpropanoic acid.

Suitable diamines and/or diols C) with anionogenic or anionic groups are compounds of the formula

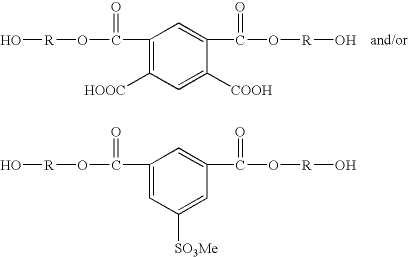

in which R is in each case a C₂-C₁₈-alkylene group and Me is Na or K.

As component C), it is also possible to use compounds of the formula

H₂N(CH₂)_{w}—NH—(CH₂)ₓ—COO⁻M⁺

H₂N(CH₂)_{w}—NH—(CH₂)ₓ—SO₃⁻M⁺

in which w and x, independently of one another, are an integer from 1 to 8, in particular 1 to 6, and M is Li, Na or K, and compounds of the formula

H₂N(CH₂CH₂O)_{y}(CH₂CH(CH₃)O)_{z}(CH₂)_{w}—NH—(CH₂)ₓ—SO₃⁻M⁺

in which w and x have the meanings given above, y and z, independently of one another, are an integer from 0 to 50, where at least one of the two variables y or z is >0. The order of the alkylene oxide units here is arbitrary. The last-mentioned compounds preferably have a number-average molecular weight in the range from about 400 to 3000. A suitable compound of this type is, for example, Poly ESP 520 from Raschig.

### Component D)

The crosslinked polyurethanes according to the invention comprise, as component D), a polyisocyanate mixture which comprises isophorone diisocyanate and hexamethylene diisocyanate. In addition, the polyisocyanate mixture can comprise at least one further polyisocyanate. Preferably, based on the total weight of the polyisocyanates used, component D) comprises 0 to 30% by weight, particularly preferably 0.1 to 15% by weight, in particular 0.5 to 5% by weight, of at least one further polyisocyanate.

Suitable further polyisocyanates D) are chosen from compounds with 2 to 5 isocyanate groups, isocyanate prepolymers with an average number of from 2 to 5 isocyanate groups, and mixtures thereof. These include, for example, aliphatic, cycloaliphatic and aromatic di-, tri- and polyisocyanates. Suitable diisocyanates D) are, for example, tetramethylene diisocyanate, 2,3,3-trimethylhexamethylene diisocyanate, 1,4-cyclohexylene diisocyanate, 1,4-phenylene diisocyanate, 2,4- and 2,6-toluoylene diisocyanate and isomer mixtures thereof (e.g. 80% 2,4-isomer and 20% 2,6-isomer), 1,5-naphthylene diisocyanate, 2,4- and 4,4′-diphenylmethane diisocyanate. A suitable triisocyanate is, for example, triphenylmethane 4,4′,4″-triisocyanate. Also suitable are isocyanate prepolymers and polyisocyanates which are obtainable by adding the abovementioned isocyanates onto polyfunctional hydroxyl- or amine-group-containing compounds. Also suitable are polyisocyanates which form through biuret, allophanate and isocyanurate formation.

Component D) particularly preferably comprises exclusively isophorone diisocyanate, hexamethylene diisocyanate, and biurets, allophanates and/or isocyanurates thereof. In particular, component D) consists of a mixture of isophorone diisocyanate and hexamethylene diisocyanate.

In component D), the quantitative weight fraction of isophorone diisocyanate is particularly preferably equally as large as or greater than the quantitative weight fraction of hexamethylene diisocyanate. In particular, in component D), the quantitative weight ratio of isophorone diisocyanate to hexamethylene diisocyanate is in a range from 3:1 to 15:1, particularly preferably from 4:1 to 10:1.

### Component E)

The crosslinked polyurethanes according to the invention comprise at least one compound E) in incorporated form which is preferably chosen from
E1) compounds with a molecular weight in a range from 60 to 286 g/mol which have two groups per molecule which are reactive toward isocyanate groups and
E2) polymers with a number-average molecular weight in the range from about 300 to 6000 which comprise two reactive groups per molecule which are reactive toward isocyanate groups;
   and mixtures thereof.

Suitable compounds E1) are, for example, diols, diamines, amino alcohols and mixtures thereof.

As component E1), preference is given to using diols whose molecular weight is in a range from about 62 to 286 g/mol. These include, for example, diols with 2 to 18 carbon atoms, preferably 2 to 10 carbon atoms, such as 1,2-ethanediol, 1,3-propanediol, 1,4-butanediol, 1,6-hexanediol, 1,5-pentanediol, 1,10-decanediol, 2-methyl-1,3-propanediol, 2,2-dimethyl-1,3-propanediol, di-, tri-, tetra-, penta- and hexaethylene glycol, neopentyl glycol, cyclohexanedimethylol and mixtures thereof. Particular preference is given to neopentyl glycol.

Preferred amino alcohols E1) are, for example, 2-aminoethanol, 2-(N-methylamino)ethanol, 3-aminopropanol, 4-aminobutanol, 1-ethylaminobutan-2-ol, 2-amino-2-methyl-1-propanol, 4-methyl-4-aminopentan-2-ol etc.

Preferred diamines E1) are, for example, ethylenediamine, propylenediamine, 1,4-diaminobutane, 1,5-diaminopentane and 1,6-diaminohexane.

The compounds specified as component E1) can be used individually or in mixtures. Particular preference is given to using 1,2-ethanediol, 1,4-butanediol, 1,6-hexanediol, neopentyl glycol, diethylene glycol, cyclohexane dimethylol and mixtures thereof.

Component E2) is preferably a polymer with a number-average molecular weight in the range from about 300 to 5000, particularly preferably about 400 to 4000, in particular 500 to 3000. Polymers E2) which can be used are, for example, polyesterdiols, polyetherols, polysiloxanes and mixtures thereof. Polyetherols are preferably polyalkylene glycols, e.g. polyethylene glycols, polypropylene glycols etc., copolymers of ethylene oxide and propylene oxide or block copolymers of ethylene oxide, propylene oxide and butylene oxide which comprise the copolymerized alkylene oxide units in random distribution or in the form of blocks. Also suitable are α,ω-diaminopolyethers which can be prepared by amination of polyalkylene oxides with ammonia. Preference is given to using polyesterdiols and mixtures which comprise these as component E2).

Preferred polyesterdiols E2) have a number-average molecular weight in the range from about 400 to 5000, preferably 500 to 3000, in particular 600 to 2000.

Suitable polyesterdiols E2) are all those which are customarily used for the preparation of polyurethane, in particular those based on aromatic dicarboxylic acids, such terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane.

Preference is given to using polyesterdiols E2) based on aromatic and aliphatic dicarboxylic acids and aliphatic diols, in particular those in which the aromatic dicarboxylic acid constitutes 10 to 95 mol %, in particular 40 to 90 mol %, of the total dicarboxylic acid content (remainder aliphatic dicarboxylic acids).

Particularly preferred polyesterdiols E2) are the reaction products of phthalic acid/diethylene glycol, isophthalic acid/1,4-butanediol, isophthalic acid/adipic acid/-1,6-hexanediol, 5-NaSO₃-isophthalic acid/phthalic acid/adipic acid/1,6-hexanediol, adipic acid/ethylene glycol, isophthalic acid/adipic acid/neopentyl glycol, isophthalic acid/adipic acid/neopentyl glycol/diethylene glycol/dimethylolcyclohexane and 5-NaSO₃-isophthalic acid/isophthalic acid/adipic acid/neopentyl glycol/diethylene glycol/dimethylolcyclohexane, isopthalic acid/adipic acid, neopentyl glycol/dimethylol-cyclohexane.

Also preferred as component E2) are polyesterdiols based on linear or branched C₈-C₃₀-di- or polycarboxylic acids and C₈-C₃₀-hydroxycarboxylic acids. Preferred carboxylic acids and hydroxycarboxylic acids are, for example, azelaic acid, dodecanedioc acid, suberic acid, pimelic acid, sebacic acid, tetradecanedioc acid, citric acid, ricinoleic acid, hydroxystearic acid and mixtures thereof. The diol components used for the preparation of these polyesterdiols are preferably 1,4-butanediol, 1,5-pentanediol, 1,6-hexanediol, neopentyl glycol, 1,4-dimethylolcyclohexane, diethylene glycol and mixtures thereof.

### Component F)

Component F) is preferably a polysiloxane with a number-average molecular weight in the range from about 300 to 100 000, for example 300 to 30 000, preferably 300 to 10 000, in particular 300 to 4000, especially 400 to 3000.

Suitable compounds of component F) are polysiloxanes of the general formula I

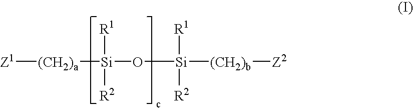

in which
a and b, independently of one another, are 1 to 8, preferably 2 to 6,
c is 2 to 100, preferably 3 to 50,
R¹ and R², independently of one another, are C₁-C₈-alkyl, benzyl, C₅-C₈-cycloalkyl or phenyl,
Z¹ and Z², independently of one another, are OH, NHR³ or a radical of the formula II

—(CH₂CH₂O)ᵥ(CH₂CH(CH₃)O)_{w}—H (II)

where
in the formula II the order of the alkylene oxide units is arbitrary and
v and w, independently of one another, are an integer from 0 to 200, where the sum of v and w is >0,
R³ is hydrogen, C₁-C₈-alkyl or C₅-C₈-cycloalkyl.

Preferably, in the compounds of the formula I, the radicals R¹ and R² are chosen, independently of one another, from methyl, ethyl, benzyl, phenyl and cyclohexyl. R¹ and R² are both particularly preferably methyl.

According to a suitable embodiment, the polysiloxanes F) of the general formula I have no alkylene oxide radicals of the general formula II. These polysiloxanes F) then preferably have a number-average molecular weight in the range from about 300 to 10 000, preferably 400 to 5000.

Suitable polysiloxanes F) which have no alkylene oxide radicals are, for example, the Tegomer® grades from Goldschmidt.

According to a further suitable embodiment, the polysiloxanes F) are silicone poly(alkylene oxide) copolymers of the formula I, where at least one or both radicals Z¹ and/or Z² are a radical of the general formula II.

Preferably, in the formula II, the sum of the v and w is chosen such that the molecular weight of the polysiloxanes F) is in a range from about 300 to 30 000.

Preferably, the total number of the alkylene oxide units of the polysiloxanes F), i.e. the sum of v and w in the formula II, is in a range from about 3 to 200, preferably 5 to 180.

Suitable silicone poly(alkylene oxide) copolymers F), which are known, for example, under the international generic name Dimethicone, are the Tegopren® grades from Goldschmidt, Belsil® 6031 and 6032 from Wacker, Silvet® L from Witco and Pluriol® ST 4005 from BASF Aktiengesellschaft.

Suitable polysiloxanes F) are also the hydroxyl-group-containing polydimethylsiloxanes described in EP-A-277 816, to which reference is hereby made.

Suitable compounds of component F) are also compounds of the general formula III

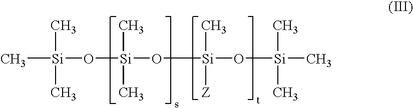

in which
the order of the siloxane units is arbitrary,
s is a value from 5 to 200, preferably 10 to 100,
Z is a radical of the formula (Z-I)=—(CH₂)ᵤ—NH₂ in which u is an integer from 1 to 10, preferably 2 to 6,
   and/or
Z is a radical of the formula (Z-II)=—(CH₂)ᵤ—NH—(CH₂)_{y}—NH₂, in which x and y, independently of one another, are 0 to 10, preferably 1 to 6, where the sum of x and y is 1 to 10, preferably 2 to 6,
t is a value from 3 to 20, preferably 3 to 10, if Z=Z-I,
t is a value from 2 to 20, preferably 2 to 10, if Z=Z-II.

These include, for example, the MAN and MAR grades from Hüls, and the Finish grades from Wacker, e.g. Finish WT 1270.

Particularly suitable polyalkylene oxide-containing silicone derivatives F) are those which comprise the following structural elements of the general formula IV:

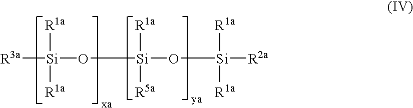

where xa and ya are integers such that the molecular weight of the polysiloxane is between 300 and 30 000,
and where the radicals R^{1a} may be identical or different, and either originate from the group of aliphatic hydrocarbons having 1 to 20 carbon atoms, are cyclic aliphatic hydrocarbons having 3 to 20 carbon atoms, or are of an aromatic nature,
and where the radicals R^{2a}, R^{3a}, R^{5a} may be identical or different and originate either from the group of aliphatic hydrocarbons having 1 to 20 carbon atoms, are cyclic aliphatic hydrocarbons having 3 to 20 carbon atoms, or are of an aromatic nature or identical to R^{6a}, where:

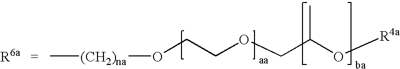

with the proviso that at least one of the radicals R^{2a}, R^{3a} or R^{5a} is a polyalkylene oxide-containing radical according to the definition of R^{6a} given above, and na is an integer from 1 to 6, in particular na=3,
aa, ba may be integers between 0 and 50, with the proviso that the sum of aa and ba is greater than 0, where

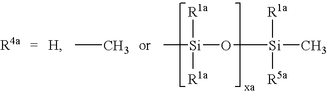

Preferably, the groups R^{1a} are chosen from the following group: methyl, ethyl, propyl, butyl, isobutyl, pentyl, isopentyl, hexyl, octyl, decyl, dodecyl and octadecyl, cycloaliphatic radicals, specifically cyclohexyl, aromatic groups, specifically phenyl or naphthyl, mixed aromatic-aliphatic radicals, such as benzyl or phenylethyl, and tolyl and xylyl.

Particular preference is given to methyl, ethyl and phenyl.

Preferably, the groups R^{2a}, R^{3a} and R^{5a} are chosen from the following group: methyl, ethyl, propyl, butyl, isobutyl, pentyl, isopentyl, hexyl, octyl, decyl, dodecyl and octadecyl, cycloaliphatic radicals, specifically cyclohexyl, aromatic groups, specifically phenyl or naphthyl, mixed aromatic-aliphatic radicals, such as benzyl or phenylethyl, and tolyl and xylyl and R⁶.

Preferred radicals R^{6a} are those in which the sum of aa+ba is between 5 and 30.

Suitable compounds of component F) are also compounds of the general formula V

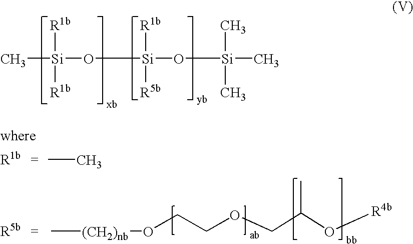

R^{4b}═—H; —COCH₃, C₁-C₄-alkyl, —OH
nb=1 to 6, preferably 2 to 4, e.g. 3,
where yb is chosen such that at least 3 OH groups are present in the molecule, i.e. for

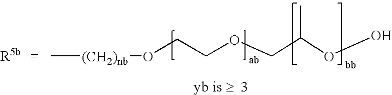

xb and yb are integers such that the molecular weight of the polysiloxane block is between 1000 and 10 000,
ab, bb may be integers between 0 and 50, with the proviso that the sum of ab and bb is greater than 0.

As component F) it is also possible to use silicone-containing polyamino compounds.

This is preferably a diaminopolyether siloxane of the formula VI, which is chosen from
polysiloxanes with repeat units of the general formula VI.1

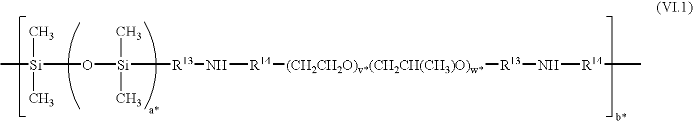

in which
a* is an integer from 0 to 100,
b* is an integer from 2 to 8,
R¹³ and R¹⁴, independently of one another, are C₁-C₈-alkylene,
the order of the alkylene oxide units is arbitrary and v* and w*, independently of one another, are an integer from 0 to 200, where the sum of v* and w* is >0,
polysiloxanes of the general formula VI.2

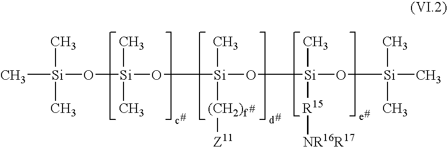

in which
R¹⁵ is a C₁-C₈-alkylene radical,
R¹⁶ and R¹⁷, independently of one another, are hydrogen, C₁-C₈-alkyl or C₅-C₈-cycloalkyl,
   the order of the siloxane units is arbitrary,
c^{#}, d^{#} and e^{#}, independently of one another, are 0 to 100, where the sum of c^{#}, d^{#} and e^{#} is at least 3, and where d^{#} and e^{#} are chosen such that at least 3 active hydrogen atoms are present in the molecule,
f^{#} is an integer from 2 to 8,
Z¹¹ is a radical of the formula VII

—R¹⁸—(CH₂CH₂O)_{g#}(CH₂CH(CH₃)O)_{h#}—H (VII)
in which
the order of the alkylene oxide units is arbitrary and g^{#} and h^{#}, independently of one another, are an integer from 0 to 200, where the sum of g^{#} and h^{#} is >O,
R¹⁸ is a C₁-C₈-alkylene radical,
   and mixtures thereof.

Polysiloxanes of the general formula VI.2 with more than two active hydrogen atoms are those in which d^{#}≧3 when R¹⁶ and R¹⁷≠H; or d^{#}+e^{#}≧3 when R¹⁶, R¹⁷═H.

Preferably, in the formula VI.1, R¹³ and R¹⁴, independently of one another, are a C₂-C₄-alkylene radical. In particular, R¹³ and R¹⁴, independently of one another, are a C₂-C₃-alkylene radical.

Preferably, the molecular weight of the compound of the formula VI.1 is in a range from about 300 to 100 000.

Preferably, in the formula VI.1, a* is an integer from 1 to 20, such as, for example, 2 to 10.

Preferably, the total number of alkylene oxide units of the compound of the formula VI.1, i.e. the sum of v* and w*, is in a range from about 3 to 200, preferably 5 to 180.

Preferably, the end-groups of the polysiloxanes with repeat units of the general formula VI.1 are chosen from (CH₃)₃SiO, H, C₁-C₈-alkyl and mixtures thereof.

Suitable alkoxylated siloxaneamines of the formula VI.1 are described, for example, in WO-A-97/32917, to the entire contents of which reference is made here. Commercially available compounds are, for example, the Silsoft® grades from Witco, e.g. Silsoft® A-843.

Preferably, in the formula VI.2, the radical R¹⁵ is a C₂-C₄-alkylene radical.

Preferably, in the formula VI.2, R¹⁸ and R¹⁷, independently of one another, are hydrogen or C₁-C₄-alkyl.

Preferably, the sum of c^{#}, d^{#} and e^{#} is chosen such that the molecular weight of the compound of the formula VI.2 is in a range from about 300 to 100 000, preferably 500 to 50 000.

Preferably, the total number of alkylene oxide units in the radical of the formula VII, i.e. the sum of g^{#} and h^{#}, is in a range from about 3 to 200, preferably 5 to 80.

Preferably, in the formula VII, the radical R¹⁸ is C₂-C₄-alkylene.

A suitable compound of the formula VI.2 is, for example, Silsoft® A-858 from Witco.

Preference is given to crosslinked polyurethanes, as described above, which are constructed from
12 to 35% by weight, preferably 18 to 30% by weight, of at least one compound of component A),
0.1 to 20% by weight, preferably 0.3 to 15% by weight, of at least one compound of component B),
5 to 20% by weight, preferably 7 to 18% by weight, of at least one compound of component C),
23 to 60% by weight, preferably 27 to 55% by weight, of at least one compound of component D),
2 to 20% by weight, preferably 3 to 15% by weight, of at least one compound of component E),
0 to 20% by weight, preferably 0.1 to 18% by weight, of at least one compound of component F),
   where the components add up to 100% by weight.

Particular preference is given to crosslinked polyurethanes which are constructed from
12 to 35% by weight, preferably 15 to 30% by weight, of at least one compound of component A),
1 to 20% by weight, preferably 3 to 15% by weight, of at least one compound of component B) which is chosen from amine-group-containing polymers with an amine number of at least 0.1 g of KOH/g and a number-average molecular weight of at least 1000,
5 to 20% by weight, preferably 7 to 18% by weight, in particular 10 to 15% by weight, of at least one compound of component C),
23 to 60% by weight, preferably 27 to 55% by weight, in particular 32 to 50% by weight, of at least one compound of component D),
2 to 20% by weight, preferably 3 to 15% by weight, in particular 5 to 12% by weight, of at least one compound of component E),
0 to 20% by weight, preferably 0 to 10% by weight (for example 0.1 to 7% by weight), of at least one compound of component F),
   where the components add up to 100% by weight.

Preference is also given to crosslinked polyurethanes, as described above, which are constructed from
15 to 35% by weight, preferably 18 to 30% by weight, of at least one compound of component A),
0.1 to 5% by weight, preferably 0.5 to 3% by weight, in particular 0.7 to 2.5% by weight, of at least one compound of component B), which is chosen from compounds which have more than two groups reactive toward NCO groups, with a molecular weight in the range from about 80 to less than 1000 g/mol, in particular trimethylolpropane,
8 to 20% by weight, preferably 10 to 18% by weight, in particular 12 to 15% by weight, of at least one compound of component C),
25 to 60% by weight, preferably 30 to 55% by weight, in particular 35 to 50% by weight, of at least one compound of component D),
3 to 20% by weight, preferably 5 to 15% by weight, in particular 7 to 12% by weight, of at least one compound of component E),
0 to 20% by weight, preferably 0 to 18% by weight (for example 0.1 to 15% by weight), of at least one compound of component F),
   where the components add up to 100% by weight.

Particular preference is given to crosslinked polyurethanes which are constructed from
A) at least one polytetrahydrofuran with two terminal hydroxyl groups per molecule and a number-average molecular weight in the range from 650 to 2000,
B) trimethylolpropane,
C) dimethylolpropanoic acid,
D) a polyisocyanate mixture which comprises isophorone diisocyanate and hexamethylene diisocyanate, and
E) neopentyl glycol.

Particular preference is also given to crosslinked polyurethanes which are constructed from
A) at least one polytetrahydrofuran with two terminal hydroxyl groups per molecule and a number-average molecular weight in the range from 650 to 2000,
B) at least one amine-group-containing polymer, in particular a copolymer of N-tert-butylaminoethyl methacrylate, vinylpyrrolidone, vinylimidazole and methacrylic acid,
C) dimethylolpropanoic acid,
D) a polyisocyanate mixture which comprises isophorone diisocyanate and hexamethylene diisocyanate, and
E) neopentyl glycol.

The polyurethanes used in the compositions according to the invention are prepared by reacting the compounds of components A), B), C), D), E) and if appropriate F). The temperature here is in a range from about 30 to 140° C., preferably about 40 to 100° C. The reaction can take place without solvent or in a suitable inert solvent or solvent mixture. Suitable solvents are aprotic polar solvents, e.g. tetrahydrofuran, ethyl acetate, N-methylpyrrolidone, dimethylformamide and preferably ketones, such as acetone and methyl ethyl ketone. Preferably, the reaction takes place under an inert gas atmosphere, such as, for example, nitrogen. The components are used in amounts such that the ratio of NCO equivalent of the compounds of component D) to equivalent of active hydrogen atom of components A), B), C), D), E) and F) is in a range from about 0.8:1 to 1.25:1, preferably 0.85:1 to 1.2:1, in particular 1.05:1 to 1.15:1. If the resulting polyurethanes also have free isocyanate groups, these are finally deactivated by adding amines, preferably amino alcohols. Suitable amino alcohols are those described above as component E), preferably 2-amino-2-methyl-1-propanol, and aqueous NaOH or aqueous KOH.

If, for the preparation of the crosslinked polyurethanes according to the invention, a component B) is used which comprises amine-group-containing polymers, then it is expedient to firstly prepare a prepolymer from these polymers of component B) and at least some of the polyisocyanates D), said prepolymer having free NCO groups, and then reacting this prepolymer, if appropriate following isolation and/or purification, with the components A), if appropriate low molecular weight compounds B), C), if appropriate still unused polyisocyanates D) and E) to give the crosslinked polyurethanes according to the invention. To prepare the prepolymer, the reaction temperature is in a range from about 30 to 100° C. If desired, for the preparation of the prepolymer, it is also possible to use a solvent or solvent mixture which has active hydrogen atoms. Preference is then given to using alcohols, such as methanol and ethanol, and mixtures of alcohols and water. Prior to reacting the prepolymer with the other components, the solvent is then preferably exchanged for one of the abovementioned aprotic-polar solvents.

The acid-group-containing polyurethanes can be converted to a water-soluble or water-dispersible form by respectively partial or complete neutralization with a base.

As a rule, the resulting salts of the polyurethanes have better solubility in water or dispersibility in water than the unneutralized polyurethanes. The bases used for neutralizing the polyurethanes may be alkali metal bases, such as sodium hydroxide solution, potassium hydroxide solution, soda, sodium hydrogencarbonate, potassium carbonate and potassium hydrogencarbonate and alkaline earth metal bases, such as calcium hydroxide, calcium oxide, magnesium hydroxide or magnesium carbonate, and ammonia and amines. Suitable amines are, for example, C₁-C₆-alkylamines, preferably n-propylamine and n-butylamine, dialkylamines, preferably diethylpropylamine and dipropylmethylamine, trialkylamines, preferably triethylamine and triisopropylamine, C₁-C₆-alkyldiethanolamines, preferably methyl- or ethyldiethanolamine and di-C₁-C₆-alkylethanolamines. For use in hair-treatment compositions, NaOH, KOH, 2-amino-2-methyl-1-propanol, diethylaminopropylamine and triisopropanolamine have proven particularly useful for neutralizing the acid-group-comprising polyurethanes. The neutralization of the polyurethanes comprising acid groups can also be carried out using mixtures of two or more bases, e.g. mixtures of sodium hydroxide solution and triisopropanolamine. Depending on the intended use, the neutralization can be partial, e.g. to 20 to 40%, or complete, i.e. to 100%.

If, in the preparation of the polyurethanes, a water-miscible organic solvent is used, this can then be removed afterward by customary processes known to the person skilled in the art, e.g. by distillation at reduced pressure. Prior to removing the solvent, water can additionally be added to the polyurethane. Replacing the solvent with water gives a solution or dispersion of the polymer from which, if desired, the polymer can be obtained in a customary manner, e.g. by spray-drying.

The polyurethanes according to the invention have K values (measured in accordance with E. Fikentscher, Cellulose-Chemie 13 (1932), pp. 58-64, on a 1% strength solution in N-methylpyrrolidone) in a range from 15 to 90, preferably 20 to 60. Their glass transition temperature is generally at least 0° C., preferably at least 20° C., particularly preferably at least 25° C. and especially at least 30° C.

The invention further provides a cosmetic or pharmaceutical composition comprising

α) at least one crosslinked polyurethane as defined above, and
β) at least one cosmetically or pharmaceutically acceptable carrier.

Preferably, the component β) is chosen from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trihydric alcohols different from iii),
v) saturated acyclic and cyclic hydrocarbons,
vi) fatty acids,
vii) fatty alcohols,
viii) propellant gases,
   and mixtures thereof.

The compositions according to the invention have, for example, an oil or fat component p) which is chosen from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes, wax esters; Vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkylbenzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils β) are, for example, linear polydimethylsiloxanes, poly(methylphenylsiloxanes), cyclic siloxanes and mixtures thereof. The number-average molecular weight of the polydimethylsiloxanes and poly(methylphenyl-siloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil and fat components β) are chosen from paraffin and paraffin oils; Vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig grease, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti, and mixtures of the abovementioned oil and fat components.

Suitable cosmetically and pharmaceutically compatible oil and fat components β) are described in Karl-Heinz Schrader, Grundlagen and Rezepturen der Kosmetika [Fundamentals and formulations of cosmetics], 2nd edition, Verlag Hüthig, Heidelberg, pp. 319-355, to which reference is hereby made.

Suitable hydrophilic carriers β) are chosen from water, 1-, 2- or polyhydric alcohols having preferably 1 to 8 carbon atoms, such as ethanol, n-propanol, isopropanol, propylene glycol, glycerol, sorbitol, etc.

The cosmetic compositions according to the invention may be skin cosmetic, hair cosmetic, dermatological, hygiene or pharmaceutical compositions. Due to their film-forming and flexible properties, the crosslinked polyurethanes described above are suitable in particular as additives for hair and skin cosmetics.

The compositions according to the invention are preferably in the form of a gel, foam, spray, an ointment, cream, emulsion, suspension, lotion, milk or paste. If desired, liposomes or microspheres can also be used.

The cosmetically or pharmaceutically active compositions according to the invention can additionally comprise cosmetically and/or dermatologically active ingredients and auxiliaries.

Preferably, the cosmetic compositions according to the invention comprise at least one crosslinked polyurethane as defined above, at least one carrier β) as defined above and at least one constituent different therefrom which is preferably chosen from cosmetically active ingredients, emulsifiers, surfactants, preservatives, perfume oils, thickeners, hair polymers, hair and skin conditioners, graft polymers, water-soluble or dispersible silicone-containing polymers, light protection agents, bleaches, gel formers, care agents, colorants, tinting agents, tanning agents, dyes, pigments, bodying agents, humectants, refatting agents, collagen, protein hydrolyzates, lipids, antioxidants, antifoams, antistats, emollients and softeners.

Customary thickeners in such formulations are crosslinked polyacrylic acids and derivatives thereof, polysaccharides and derivatives thereof, such as xanthan gum, agar agar, alginates or Tyloses, cellulose derivatives, e.g. carboxymethylcellulose or hydroxycarboxymethylcellulose, fatty alcohols, monoglycerides and fatty acids, polyvinyl alcohol and polyvinylpyrrolidone.

Suitable cosmetically and/or dermatologically active ingredients are, for example, coloring active ingredients, skin and hair pigmentation agents, tinting agents, tanning agents, bleaches, keratin-hardening substances, antimicrobial active ingredients, light filter active ingredients, repellent active ingredients, substances with hyperemic activity, substances with keratolytic and keratoplastic activity, antidandruff active ingredients, antiphlogistics, substances which have a keratinizing effect, substances which act as antioxidants or as free-radical scavengers, skin moisturizers or humectants, refatting active ingredients, deodorizing active ingredients, sebostatic active ingredients, plant extracts, antierythematous or antiallergic active ingredients and mixtures thereof.

Artificially skin-tanning active ingredients which are suitable for tanning the skin without natural or artificial irradiation with UV rays are, for example, dihydroxyacetone, alloxan and walnut shell extract. Suitable keratin-hardening substances are usually active ingredients as are also used in antiperspirants, such as, for example, potassium aluminum sulfate, aluminum hydroxychloride, aluminum lactate, etc. Antimicrobial active ingredients are used in order to destroy microorganisms or to inhibit their growth and thus serve both as preservatives and also as a deodorizing substance which reduces the formation or the intensity of body odor. These include, for example, customary preservatives known to the person skilled in the art, such as p-hydroxy-benzoates, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable light filter active ingredients are substances which absorb UV rays in the UV-B and/or UV-A region. Suitable UV filters are, for example, 2,4,6-triaryl-1,3,5-triazines in which the aryl groups may each carry at least one substituent which is preferably chosen from hydroxyl, alkoxy, specifically methoxy, alkoxycarbonyl, specifically methoxycarbonyl and ethoxycarbonyl and mixtures thereof. Also suitable are p-aminobenzoates, cinnamates, benzophenones, camphor derivatives, and pigments which stop UV rays, such as titanium dioxide, talc and zinc oxide. Suitable repellent active ingredients are compounds which are able to drive away or repel certain animals, in particular insects, from humans. These include, for example, 2-ethyl-1,3-hexanediol, N,N-diethyl-m-toluamide etc. Suitable substances with hyperemic activity which stimulate blood flow through the skin are, for example, ethereal oils, such as dwarf pine, lavender, rosemary, juniper berry, horsechestnut extract, birch leaf extract, hayseed extract, ethyl acetate, camphor, menthol, peppermint oil, rosemary extract, eucalyptus oil, etc. Suitable keratolytically and keratoplastically active substances are, for example, salicylic acid, calcium thioglycolate, thioglycolic acid and salts thereof, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics which counter skin irritations are, for example, allantoin, bisabolol, Dragosantol, camomile extract, panthenol, etc.

The cosmetic compositions according to the invention can comprise, as cosmetic and/or pharmaceutical active ingredient (and also optionally as auxiliary), at least one cosmetically or pharmaceutically acceptable polymer which differs from the crosslinked polyurethanes according to the invention. Very generally, these include anionic, cationic, amphoteric and neutral polymers.

Examples of anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes, e.g. Luviset PUR® from BASF, and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and if appropriate further vinyl esters (e.g. Luviset® grades), maleic anhydride copolymers, if appropriate reacted with alcohol, anionic polysiloxanes, e.g. carboxyfunctional, t-butyl acrylate, methacrylic acid (e.g. Luviskol® VBM), copolymers of acrylic acid and methacrylic acid with hydrophobic monomers, such as, for example, C₄-C₃₀-alkyl esters of meth(acrylic acid), C₄-C₃₀-alkyl vinyl esters, C₄-C₃₀-alkyl vinyl ethers and hyaluronic acid. Examples of anionic polymers are also vinyl acetate/crotonic acid copolymers, as are available commercially, for example, under the names Resyn® (National Starch) and Gafset® (GAF), and vinylpyrrolidone/vinyl acrylate copolymers, obtainable, for example, under the trade name Luviflex® (BASF). Other suitable polymers are the vinylpyrrolidone/acrylate terpolymer available under the name Luviflex® VBM-35 (BASF), and sodium sulfonate-containing polyamides or sodium sulfonate-containing polyesters. Also suitable are vinylpyrrolidone/ethyl methacrylate/methacrylic acid copolymers, as are sold by Stepan under the names Stepanhold-Extra and -R1 and the Carboset® grades from BF Goodrich.

Suitable cationic polymers are, for example, cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinyl-pyrrolidone/dimethylaminoethyl methacrylate, quaternized with diethyl sulfate (Luviquat® PQ 11), copolymers of N-vinylcaprolactam/N-vinylpyrrolidone/N-vinyl-imidazolium salts (Luviquat® Hold); cationic cellulose derivatives (Polyquaternium-4 and -10), acrylamido copolymers (Polyquaternium-7) and chitosan. Suitable cationic (quaternized) polymers are also Merquat® (polymer based on dimethyldiallyl-ammonium chloride), Gafquat® (quaternary polymers which are formed by the reaction of polyvinylpyrrolidone with quaternary ammonium compounds), Polymer JR (hydroxyethylcellulose with cationic groups) and vegetable-based cationic polymers, e.g. guar polymers, such as the Jaguar® grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF), or polyvinyl-pyrrolidone and copolymers thereof, in particular with vinyl esters, such as vinyl acetate, e.g. Luviskol® VA 37 (BASF); polyamides, e.g. those based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and the alkali metal and ammonium salts thereof are preferred zwitterionic polymers. Other suitable zwitterionic polymers are methacroylethylbetaine/-methacrylate copolymers, which are available commercially under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyether siloxanes, such as Tegopren® (Goldschmidt) or Belsil® (Wacker).

The formulation base of pharmaceutical compositions according to the invention preferably comprises pharmaceutically acceptable auxiliaries. Pharmaceutically acceptable auxiliaries are the auxiliaries which are known for use in the fields of pharmacy, food technology and related fields, in particular the auxiliaries listed in the relevant pharmacopeias (e.g. DAB Ph. Eur. BP NF), and other auxiliaries whose properties do not preclude a physiological application.

Suitable auxiliaries may be: lubricants, wetting agents, emulsifying and suspending agents, preservatives, antioxidants, anti-irritative substances, chelating agents, emulsion stabilizers, film formers, gel formers, odor-masking agents, resins, hydrocolloids, solvents, solubility promoters, neutralizing agents, permeation accelerators, pigments, quaternary ammonium compounds, refatting and superfatting agents, ointment bases, cream bases or oil bases, silicone derivatives, stabilizers, sterilizing agents, propellants, drying agents, opacifiers, thickeners, waxes, softeners, white oils. Formulation in this regard is based on expert knowledge, as given, for example, in Fiedler, H. P. Lexikon der Hilfsstoffe für Pharmazie, Kosmetik und angrenzende Gebiete [Lexicon of auxiliaries for pharmacy, cosmetics and related fields], 4th Ed., Aulendorf: ECV-Editio-Kantor-Verlag, 1996.

To prepare the dermatological compositions according to the invention, the active ingredients can be mixed or diluted with a suitable auxiliary (excipient). Excipients can be solid, semisolid or liquid materials which can serve as vehicles, carriers or medium for the active ingredient. The admixing of further auxiliaries is carried out, where desired, in the manner known to the person skilled in the art. The crosslinked polyurethanes are also suitable as auxiliaries in pharmacy, preferably as or in (a) coating(s) or (a) binder(s) for solid drug forms. They also be used in creams and as tablet coatings and tablet binders.

In a preferred embodiment, the compositions according to the invention are skin-cleansing compositions.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and shower gels, foam baths, oil baths and scrub preparations, shaving foams, lotions and creams.

According to a further preferred embodiment, the compositions according to the invention are cosmetic compositions for the care and protection of the skin, nail care compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage make-up, mascara and eyeshadows, lipsticks, kohl pencils, eyeliners, blushers, powders and eyebrow pencils.

The crosslinked polyurethanes can also be used in nose strips for pore cleansing, in antiacne compositions, repellents, shaving compositions, depilatories, intimate care compositions, footcare compositions and in babycare.

The skincare compositions according to the invention are, in particular, W/O or O/W skin creams, day creams and night creams, eye creams, face creams, antiwrinkle creams, moisturizing creams, bleaching creams, vitamin creams, skin lotions, care lotions and moisturizing lotions.

Skin cosmetic and dermatological compositions based on the above-described crosslinked polyurethanes exhibit advantageous effects. The polymers can, inter alia, contribute to the moisturizing, and conditioning of the skin and to an improvement in the feel of the skin. The polymers can also act as thickeners in the formulations. By adding the polymers according to the invention, it is possible to achieve a considerable improvement in skin compatibility in certain formulations.

Skin cosmetic and dermatological compositions preferably comprise at least one crosslinked polyurethane in an amount of from about 0.001 to 30% by weight, preferably 0.01 to 20% by weight, very particularly preferably 0.1 to 12% by weight, based on the total weight of the composition.

Light protection agents based on the crosslinked polyurethanes, in particular, have the property of increasing the residence time of the UV-absorbing ingredients compared with customary auxiliaries such as polyvinylpyrrolidone.

Depending on the field of use, the compositions according to the invention can be applied in a form suitable for skin care, such as, for example, as cream, foam, gel, pencil, mousse, milk, spray (pump spray or spray containing propellant) or lotion.

As well as comprising the crosslinked polyurethanes and suitable carriers, the skin cosmetic preparations can also comprise further active ingredients and auxiliaries customary in skin cosmetics, as described above. These include, preferably, emulsifiers, preservatives, perfume oils, cosmetic active ingredients, such as phytantriol, vitamins A, E and C, retinol, bisabolol, panthenol, light protection agents, bleaches, colorants, tinting agents, tanning agents, collagen, protein hydrolyzates, stabilizers, pH regulators, dyes, salts, thickeners, gel formers, bodying agents, silicones, moisturizers, refatting agents and further customary additives.

Preferred oil and fatty components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymers according to the invention can also be mixed with traditional polymers where specific properties are to be set.

To set certain properties, such as, for example, improving the feel to the touch, the spreading behavior, the water resistance and/or the binding of active ingredients and auxiliaries, such as pigments, the skin cosmetic and dermatological preparations can additionally also comprise conditioning substances based on silicone compounds. Suitable silicone compounds are, for example, polyalkyl siloxanes, polyaryl siloxanes, polyarylalkyl siloxanes, polyether siloxanes or silicone resins.

The cosmetic or dermatological preparations are prepared by customary methods known to the person skilled in the art.

The cosmetic and dermatological compositions are preferably in the form of emulsions, in particular water-in-oil (W/O) emulsions or oil-in-water (O/W) emulsions. It is, however, also possible to choose other types of formulation, for example hydrodispersions, gels, oils, oleogels, multiple emulsions, for example in the form of W/O/W or O/W/O emulsions, anhydrous ointments or ointment bases, etc.

The emulsions are prepared by known methods. Apart from at least one crosslinked polyurethane the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The choice of emulsion type-specific additives and the preparation of suitable emulsions is described, for example, in Schrader, Grundlagen und Rezepturen der Kosmetika [Fundamentals and Formulations of Cosmetics], Hüthig Buch Verlag, Heidelberg, 2nd Edition, 1989, third part, to which express reference is made here.

A suitable emulsion, e.g. for a skin cream etc., generally comprises an aqueous phase which is emulsified by means of a suitable emulsifier system in an oil or fatty phase. To provide the aqueous phase, a crosslinked polyurethane can be used.

Preferred fatty components which the fatty phase of the emulsions may comprise are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start-point under atmospheric pressure is about 250° C. and whose distillation end-point is 410° C., such as, for example, vaseline oil; esters of saturated or unsaturated fatty acids, such as alkyl myristates, e.g. i-propyl, butyl or cetyl myristate, hexadecyl stearate, ethyl or i-propyl palmitate, octanoic or decanoic acid triglycerides and cetyl ricinoleate.

The fatty phase may also comprise silicone oils soluble in other oils, such as dimethylpolysiloxane, methylphenylpolysiloxane and the silicone glycol copolymer, fatty acids and fatty alcohols.

Besides the crosslinked polyurethanes, it is also possible to use waxes, such as, for example, carnauba wax, candellila wax, beeswax, microcrystalline wax, ozokerite wax and the oleates, myristates, linoleates and stearates of Ca, Mg and Al.

In addition, an emulsion according to the invention may be in the form of an O/W emulsion. Such an emulsion usually comprises an oil phase, emulsifiers which stabilize the oil phase in the water phase and an aqueous phase which is usually present in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one crosslinked polyurethane and customary anionic surfactants as base surfactants and amphoteric and/or nonionic surfactants as cosurfactants. Further suitable active ingredients and/or auxiliaries are generally chosen from lipids, perfume oils, dyes, organic acids, preservatives and antioxidants, and thickeners/gel formers, skin conditioning agents and humectants.

These formulations preferably comprise 2 to 50% by weight, preferably 5 to 40% by weight, particularly preferably 8 to 30% by weight, of surfactants, based on the total weight of the formulation.

All anionic, neutral, amphoteric or cationic surfactants customarily used in body-cleansing compositions can be used in the washing, shower and bath preparations.

Suitable anionic surfactants are, for example, alkyl sulfates, alkyl ether sulfates, alkylsulfonates, alkylarylsulfonates, alkyl succinates, alkyl sulfosuccinates, N-alkoyl-sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropyl-betaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, ethoxylated fatty acid amides, alkyl polyglycosides or sorbitan ether esters.

The washing, shower and bath preparations can also comprise customary cationic surfactants such as, for example, quaternary ammonium compounds, for example cetyltrimethylammonium chloride.

The shower gel/shampoo formulations can further comprise thickeners, such as, for example, sodium chloride, PEG-55, propylene glycol oleate, PEG-120 methyl glucose dioleate and others, and also preservatives, further active ingredients and auxiliaries and water.

In a preferred embodiment, the compositions according to the invention are hair-treatment compositions.

Hair-treatment compositions according to the invention preferably comprise at least one crosslinked polyurethane in an amount in the range from about 0.1 to 30% by weight, preferably 0.5 to 20% by weight, based on the total weight of the composition.

The hair-treatment compositions according to the invention are preferably in the form of a setting foam, hair mousse, hair gel, shampoo, hairspray, hair foam, end fluid neutralizers for permanent waves, hair colorants and bleaches or “hot-oil treatments”. Depending on the field of use, the hair cosmetic preparations can be applied in the form of an (aerosol) spray, (aerosol) foam, gel, gel spray, cream, lotion or wax. Hairsprays comprise both aerosol sprays and also pump sprays without propellent gas. Hair foams comprise both aerosol foams and also pump foams without propellent gas. Hairsprays and hair foams preferably comprise predominantly or exclusively water-soluble or water-dispersible components. If the compounds used in the hairsprays and hair foams according to the invention are water-dispersible, they can be used in the form of aqueous microdispersions with particle diameters of usually 1 to 350 nm, preferably 1 to 250 nm. The solids contents of these preparations are usually in a range from about 0.5 to 20% by weight. These microdispersions generally require no emulsifiers or surfactants for their stabilization.

In a preferred embodiment, the hair cosmetic formulations according to the invention comprise

a) 0.05 to 20% by weight of at least one crosslinked polyurethane,
b) 20 to 99.95% by weight of water and/or alcohol,
c) 0 to 50% by weight of at least one propellent gas,
d) 0 to 5% by weight of at least one emulsifier,
e) 0 to 3% by weight of at least one thickener, and
f) up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances such as panthenol, collagen, vitamins, protein hydrolyzates, alpha- and beta-hydroxycarboxylic acids, stabilizers, pH regulators, dyes, viscosity regulators, gel formers, salts, humectants, refatting agents, complexing agents and further customary additives.

Also included are all styling and conditioner polymers known in cosmetics which can be used in combination with the polymers according to the invention if quite specific properties are to be set.

Suitable conventional hair cosmetic polymers are, for example, the abovementioned cationic, anionic, neutral, nonionic and amphoteric polymers, to which reference is made here.

To set certain properties, the preparations can also additional comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and aminofunctional silicone compounds such as amodimethicone (CTFA).

The polymers according to the invention are particularly suitable as setting agents in hair styling preparations, in particular hairsprays (aerosol sprays and pump sprays without propellent gas) and hair foams (aerosol foams and pump foams without propellent gas).

In a preferred embodiment, spray preparations comprise

a) 0.1 to 10% by weight of at least one crosslinked polyurethane,
b) 20 to 99.9% by weight of water and/or alcohol,
c) 0 to 70% by weight of at least one propellant,
d) 0 to 20% by weight of further constituents.

Propellants are the propellants used customarily for hairsprays or aerosol foams. Preference is given to mixtures of propane/butane, pentane, dimethyl ether, 1,1-difluororethane (HFC-152 a), carbon dioxide, nitrogen or compressed air.

A formulation preferred according to the invention for aerosol hair foams comprises

a) 0.1 to 10% by weight of at least one crosslinked polyurethane,
b) 55 to 99.8% by weight of water and/or alcohol,
c) 5 to 20% by weight of a propellant,
d) 0.1 to 5% by weight of an emulsifier,
e) 0 to 10% by weight of further constituents.

The emulsifiers used may be all emulsifiers customarily used in hair foams. Suitable emulsifiers may be nonionic, cationic or anionic or amphoteric.

Examples of nonionic emulsifiers (INCI nomenclature) are Laureths, e.g. Laureth-4; ceteths, e.g. Ceteth-1, polyethylene glycol cetyl ether; ceteareths, e.g. Ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, Quaternium-1 to x (INCI).

Anionic emulsifiers may be chosen, for example, from the group of alkyl sulfates, alkyl ether sulfates, alkylsulfonates, alkylarylsulfonates, alkyl succinates, alkyl sulfo-succinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
a) 0.1 to 10% by weight of at least one crosslinked polyurethane,
b) 80 to 99.9% by weight, preferably 80 to 99.85% by weight of water and/or alcohol,
c) 0 to 3% by weight, preferably 0.05 to 2% by weight, of a gel former,
d) 0 to 20% by weight of further constituents.

In the preparation of gels based on the crosslinked polyurethanes according to the invention, customary gel formers can be used, for example, in order to establish special rheological or other application properties of the gels. Gel formers which may be used are all gel formers customary in cosmetics. These include slightly crosslinked polyacrylic acid, for example Carbomer (INCI), cellulose derivates, e.g. hydroxypropylcellulose, hydroxyethylcellulose, cationically modified celluloses, polysaccharides, e.g. xanthan gum, caprylic/capric triglyceride, sodium acrylate, copolymers, Polyquaternium-32 (and) Paraffinum Liquidum (INCI), sodium acrylate copolymers (and) Paraffinum Liquidum (and) PPG-1 Trideceth-6, acrylamidopropyl-trimonium chloride/acrylamide copolymers, Steareth-10 allyl ether acrylate copolymers, Polyquaternium-37 (and) Paraffinum Liquidum (and) PPG-1 Trideceth-6, Polyquaternium 37 (and) propylene glycol dicaprate dicaprylate (and) PPG-1 Trideceth-6, Polyquaternium-7, Polyquaternium-44. Crosslinked homopolymers of acrylic acid suitable as gel formers are commercially available for example under the name Carbopol® from BF GOODRICH. Preference is also given to hydrophobically modified crosslinked polyacrylate polymers, such as Carbopol® Ultrez 21 from Noveon. Further examples of anionic polymers suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm & Haas.

The crosslinked polyurethanes according to the invention can be used as conditioners in cosmetic preparations.

The crosslinked polyurethanes according to the invention can preferably be used in shampoo formulations as setting agents and/or conditioners. Preferred shampoo formulations comprise

a) 0.05 to 10% by weight of at least one crosslinked polyurethane,
b) 25 to 94.95% by weight of water,
c) 5 to 50% by weight of surfactants,
d) 0 to 5% by weight of a further conditioner,
e) 0 to 10% by weight of further cosmetic constituents.

In the shampoo formulations it is possible to use all anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropylbetaines, alkylsulfobetaines, alkyl glycinates, alkyl carboxyglycinates, alkyl amphoacetates or propionates, alkyl amphodiacetates or dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Alkylamine oxides, mono- or dialkylalkanolamides, fatty acid esters of polyethylene glycols, alkyl polyglycosides or sorbitan ether esters are also suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioners can be used in combination with the crosslinked polyurethanes. These include, for example, the abovementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinyl-imidazolium salts (Luviquat® Hold); cationic cellulose derivatives (Polyquaternium-4 and -10), acrylamide copolymers (Polyquaternium-7). It is also possible to use protein hydrolyzates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds, such as amodimethicone (CTFA). It is also possible to use cationic guar derivatives, such as Guar Hydroxypropyltrimonium Chloride (INCI).

The invention further provides the use of a crosslinked polyurethane, as defined above, as auxiliary in pharmacy, preferably as or in (a) coating(s) for solid drug forms, for modifying rheological properties, as surface-active compound, as or in (an) adhesive(s), and as or in (a) coating(s) for the textile, paper, printing and leather industry.

The invention is explained in more detail by reference to the following nonlimiting examples.

### EXAMPLES

### I. Preparation of Crosslinked Polyurethanes

### Example 1

In a 4-necked flask which was equipped with stirrer, dropping funnel, thermometer, reflux condenser and a device for working under nitrogen, 500 g (0.5 mol) of polytetrahydrofuran (Mₙ=1000 g/mol), 33.5 g (0.25 mol) of trimethylolpropane, 335 g (2.5 mol) of dimethylolpropanoic acid, 176.8 g (1.7 mol) of neopentyl glycol and 0.85 g of 1,4-diazabicyclo[2.2.2]octane (DABCO) were dissolved in 690 g of methyl ethyl ketone with heating to a temperature of 80° C. and with stirring. After all of the components had dissolved, the reaction mixture was left to cool to about 60° C. Then, at this temperature and with stirring, a mixture of 134.4 g (0.8 mol) of hexamethylene diisocyanate and 932.4 g (4.2 mol) of isophorone diisocyanate was metered in such that the reaction temperature remained below 90° C. The reaction mixture was then after-stirred for a further 3 to 4 hours at 80° C. until the NCO content of the mixture remained virtually constant. After cooling to 40° C., 405 g (2.375 mol) of a 50% strength 2-amino-2-methyl-1-propanol solution were added to the reaction mixture, and then the solvent was removed under reduced pressure at 40° C. Following steam treatment, the resulting product was admixed with water, giving a 25% strength by weight essentially clear microdispersion. Pulverulent crosslinked polyurethanes can be isolated by spray-drying.

The crosslinked polyurethanes 2 to 5 were prepared analogously.

### Example 8

In a 4-necked flask which was equipped with stirrer, dropping funnel, thermometer, reflux condenser and a device for working under nitrogen, 500 g (0.5 mol) of polytetrahydrofuran (Mₙ=1000 g/mol), 33.5 g (0.25 mol) of trimethylolpropane, 335 g (2.5 mol) of dimethylolpropanoic acid, 176.8 g (1.7 mol) of neopentyl glycol and 0.85 g of DABCO were dissolved in 690 g of methyl ethyl ketone with heating to a temperature of 80° C. and with stirring. As soon as all of the components had dissolved, the reaction mixture was left to cool to 60° C. Then, at this temperature, and with stirring, a mixture of 134.4 g (0.8 mol) of hexamethylene diisocyanate and 932.4 g (4.2 mol) of isophorone diisocyanate was metered in such that the reaction temperature remained below 90° C. The reaction mixture was then after-stirred for about a further three hours at about 80° C. until the NCO content remained virtually constant and the mixture was then left to cool to a temperature of 40° C. The reaction product was admixed with 467 g (2.5 mol) of 30% strength KOH solution and then the solvent was distilled off under reduced pressure at 40° C. After steam treatment, the mixture was diluted with water, giving a 25% strength by weight essentially clear microdispersion. Pulverulent products can be obtained by spray-drying.

The crosslinked polyurethanes 9 to 12 were prepared analogously.

### Example 14

In a 4-necked flask which was equipped with stirrer, dropping funnel, thermometer, reflux condenser and a device for working under nitrogen, 500 g (0.5 mol) of polytetrahydrofuran (Mₙ=1000 g/mol), 335 g (2.5 mol) of dimethylolpropanoic acid, 176.8 g (1.7 mol) of neopentyl glycol and 0.85 g of DABCO were dissolved in 690 g of methyl ethyl ketone with heating to a temperature of 80° C. and with stirring. As soon as all of the components had dissolved, the reaction mixture was left to cool to 60° C. and, at this temperature and with stirring, a mixture of 134.4 g (0.8 mol) of hexamethylene diisocyanate and 932.4 g (4.2 mol) of isophorone diisocyanate was metered in slowly such that the reaction temperature remained below 90° C. The reaction mixture was then after-stirred at 80° C. for about three hours until the NCO content of the mixture remained virtually constant. The mixture was then left to cool to a temperature of 40° C., and 346.5 g of an aqueous 30% strength amino-group-containing copolymer solution (vinylpyrrolidone/vinylimidazole/methacrylic acid/N-tert-butylaminoethyl methacrylate copolymer) were added and the mixture was stirred for a further 30 minutes at 40° C. The reaction product was then neutralized with 467 g (2.5 mol) of 30% strength aqueous KOH solution and the solvent was removed under reduced pressure at 40° C. After steam treatment, the reaction product was diluted, giving a 25% strength by weight dispersion. Pulverulent products can be obtained by spray-drying.

The crosslinked polyurethanes 6, 7 and 14 were prepared analogously.

| | P (THF | | Pol.-amine | | | | | | | |
|---|---|---|---|---|---|---|---|---|---|---|
| | 1000) | TMP | [% by | DMPA | NPG | IPDI | HDI | KOH | AMP | K value |
| | mol | [mol] | wt.*)] | [mol] | [mol] | [mol] | [mol] | [D.N. %] | [D.N. %] | (1% NMP) |
| 1 | 0.5 | 0.25 | — | 2.5 | 1.7 | 4.2 | 0.8 | — | 95 | 35.6 |
| 2 | 0.5 | 0.35 | — | 2.5 | 1.7 | 4.1 | 0.8 | — | 95 | 37.9 |
| 3 | 0.5 | 0.35 | — | 2.5 | 1.7 | 4.0 | 1.0 | — | 95 | 38.1 |
| 4 | 0.5 | 0.40 | — | 2.5 | 1.7 | 4.0 | 1.0 | — | 95 | 39.8 |
| 5 | 0.5 | 0.45 | — | 2.5 | 1.6 | 4.0 | 1.0 | — | 95 | 38.5 |
| 6 | 0.5 | — | 5 | 2.5 | 1.7 | 4.0 | 1.0 | — | 95 | 37.5 |
| 7 | 0.5 | — | 5 | 2.5 | 1.7 | 4.2 | 0.8 | — | 95 | 36.8 |
| 8 | 0.5 | 0.25 | — | 2.5 | 1.7 | 4.2 | 0.8 | 100 | — | 39.1 |
| 9 | 0.5 | 0.35 | — | 2.5 | 1.7 | 4.1 | 0.9 | 100 | — | 41.3 |
| 10 | 0.5 | 0.40 | — | 2.5 | 1.7 | 4.0 | 1.0 | 100 | — | 43.9 |
| 11 | 0.5 | 0.40 | — | 2.5 | 1.7 | 4.0 | 1.0 | 100 | — | 44.6 |
| 12 | 0.5 | 0.45 | — | 2.5 | 1.6 | 4.0 | 1.0 | 100 | — | 46.8 |
| 13 | 0.5 | — | 5 | 2.5 | 1.7 | 4.0 | 1.0 | 100 | — | 40.6 |
| 14 | 0.5 | — | 5 | 2.5 | 1.7 | 4.2 | 0.8 | 100 | — | 38.7 |

P(THF)1000 = polytetrahydrofuran, MW = 1000 g/mol
TMP = trimethylolpropane
Pol.-amine = P(VP/VI/MAA/NtBAEMA)
*)= Pol.-amine is used in weight based on the total solids,
NPG = neopentyl glycol
DMPA = dimethylolpropanoic acid
IPDI = isophorone diisocyanate
HDI = hexamethylene diisocyanate
AMP = 2-amino-2-methyl-1-propanol
KOH = potassium hydroxide
D.N. % = degree of neutralization
NMP = N-methyl-pyrrolidone

### II. APPLICATION EXAMPLES

### Example 1

| VOC 80 aerosol hairspray | [%] |
|---|---|
| Polymer from example No. 1 (25% strength solution) | 12.00 |
| Water | 8.00 |
| Dimethyl ether | 40.00 |
| Ethanol | 40.00 |

Further addition: silicone, perfume, antifoam . . . .

The example can be repeated with the crosslinked polyurethanes of examples 2, 3, 4, 5, 6, 7, 8, 9, 10, 11, 12, 13 and 14. In every case, a VOC 80 aerosol hairspray with good properties is obtained.

### Example 2

| VOC 55 aerosol hairspray | [%] |
|---|---|
| Polymer from example No. 1 (25% strength solution) | 12.00 |
| Water | 33.00 |
| Dimethyl ether | 40.00 |
| Ethanol | 15.00 |

further addition: silicone, perfume, antifoam . . .

The example can be repeated with the crosslinked polyurethanes from examples 2, 3, 4, 5, 6, 7, 8, 9, 10, 11, 12, 13 and 14. In every case, a VOC 55 aerosol hairspray with good properties is obtained.

### Example 3

| VOC 55 aerosol hairspray | [%] |
|---|---|
| Polymer from example No. 1 (25% strength solution) | 10.00 |
| Ultrahold Strong (BASF) | 1.00 |
| Water | 34.00 |
| Dimethyl ether | 40.00 |
| Ethanol | 15.00 |
| +AMP | to pH 8.3 |

further addition: silicone, perfume, antifoam . . .

The example can be repeated with the crosslinked polyurethanes from examples 2, 3, 4, 5, 6, 7, 8, 9, 10, 11, 12, 13 and 14. In every case, a VOC 55 aerosol hairspray with good properties is obtained.

### Example 4

| VOC 55 aerosol hairspray | [%] |
|---|---|
| Polymer from example No. 1 (25% strength solution) | 8.00 |
| Stepanhold R-1*⁾ (Stepan Chemical Co.) | 1.00 |
| Water | 36.00 |
| Dimethyl ether | 40.00 |
| Ethanol | 15.00 |
| +AMP | to pH 8.3 |

further addition: silicone, perfume, antifoam . . .
*⁾Stepanhold R-1 = poly(vinylpyrrolidone/ethyl methacrylat/methacrylic acid)

The example can be repeated with the crosslinked polyurethanes from examples 2, 3, 4, 5, 6, 7, 8, 9, 10, 11, 12, 13 and 14. In every case, a VOC 55 aerosol hairspray with good properties is obtained.

### Example 5

| VOC 55 hand pump spray | [%] |
|---|---|
| Polymer from example No. 1 (25% strength solution) | 12.00 |
| Water | 33.00 |
| Ethanol | 55.00 |

further addition: silicone, perfume, antifoam . . .

The example can be repeated with the crosslinked polyurethanes from examples 2, 3, 4, 5, 6, 7, 8, 9, 10, 11, 12, 13 and 14. In every case, a VOC 55 hand pump spray with good properties is obtained.

### Example 6

| Aqueous handpump spray | [%] |
|---|---|
| Polymer from example No. 1 (25% strength solution) | 10.00 |
| Luviset Clear*⁾ (20% strength solution) | 5.00 |
| Water | 85.00 |

further addition: water-soluble silicone, perfume, antifoam . . .
*⁾Luviset Clear: poly(vinylpyrrolidone/methacrylamide/vinylimidazol), BASF

The example can be repeated with the crosslinked polyurethanes from examples 2, 3, 4, 5, 6, 7, 8, 9, 10, 11, 12, 13 and 14. In every case, an aqueous hand pump spray with good properties is obtained.

### Example 7

| Foam conditioner | [%] |
|---|---|
| Polymer from example No. 1 (25% strength aqueous solution) | 20.00 |
| Cremophor A 25 (Ceteareth 25/BASF) | 0.2 |
| Comperlan KD (Coamide DEA/Henkel) | 0.1 |
| Water | 69.7 |
| Propane/butane | 10.0 |

further addition: perfume, preservative . . .

Preparation: weigh in and dissolve with stirring. Bottle and add propellent gas.

The example can be repeated with the crosslinked polyurethanes from examples 2, 3, 4, 5, 6, 7, 8, 9, 10, 11, 12, 13 and 14. In every case, a foam conditioner with good properties is obtained.

### Example 8

| Hair gel containing Aculyn 28: | [%] |
|---|---|
| Phase 1: | |
| Polymer from example No. 1 (25% strength aqueous solution) | 12.00 |
| Water, dist. | 37.00 |
| Aminomethylpropanol (38% strength solution) | 1.0 |

further addition: preservative, soluble ethoxylated silicone, perfume . . .

| | |
|---|---|
| Phase 2: | |
| Aculyn 28 (1% strength aqueous suspension) | 50.00 |

### Preparation:

Phases 1 and 2 are weighed in separately and homogenized. Phase 2 is then slowly stirred into phase 1. An essentially clear, stable gel is formed.

The example can be repeated with the crosslinked polyurethanes from examples 2, 3, 4, 5, 6, 7, 8, 9, 10, 11, 12, 13 and 14. In every case, a hair gel containing Aculyn 28 with good properties is obtained.

### Example 9

| Hair gel containing hydroxyethylcellulose: | [%] |
|---|---|
| Phase 1: | |
| Polymer from example No. 1 (25% strength solution) | 12.00 |
| Water, dist. | 30.00 |

further addition: preservative, soluble ethoxylated silicone, perfume . . .
Phase 2:

| | |
|---|---|
| Natrosol HR 250 (5% strength solution) | 50.00 |
| hydroxyethylcellulose (Hercules) | |

### Preparation:

Phases 1 and 2 are weighed in separately and homogenized. Phase 2 is then slowly stirred into phase 1. An essentially clear, stable gel is formed.

The example can be repeated with the crosslinked polyurethanes from examples 2, 3, 4, 5, 6, 7, 8, 9, 10, 11, 12, 13 and 14. In every case, a hair gel containing hydroxyethylcellulose with good properties is obtained.

### Example 10

| Conditioner shampoo: | | [%] |
|---|---|---|
| A) | Texapon NSO 28% strength (sodium laureth sulfate/Henkel) | 50.00 |
| | Comperlan KS (Coamide DEA/Henkel) | 1.00 |
| | Polymer from example No. 1 (25% strength aqueous solution) | 20.00 |
| | Perfume oil | q.s. |
| B) | Water | 27.5 |
| | Sodium chloride | 1.5 |
| | Preservative . . . | q.s. |

### Preparation:

Phases 1 and 2 are weighed in separately and homogenized. Phase 2 is then slowly stirred into phase 1. An essentially clear, stable gel is formed.

The example can be repeated with the crosslinked polyurethanes from examples 2, 3, 4, 5, 6, 7, 8, 9, 10, 11, 12, 13 and 14. In every case, a conditioner shampoo with good properties is obtained.

### Example 11

Standard O/W cream:

| | [%] | CTFA name |
|---|---|---|
| Oil phase: | | |
| Cremophor A6 | 3.5 | ceteareth-6 (and) stearyl alcohol |
| Cremophor A25 | 3.5 | ceteareth-25 |
| Glycerol monostearate s.e. | 2.5 | glyceryl stearate |
| Paraffin oil | 7.5 | paraffin oil |
| Cetyl alcohol | 2.5 | cetyl alcohol |
| Luvitol EHO | 3.2 | cetearyl octanoate |
| Vitamin E acetate | 1.0 | tocopheryl acetate |
| Nip-Nip | 0.1 | methyl and propyl |
| | | 4-hydroxybenzoate (7:3) |
| Water phase: | | |
| Polymer from example No. 1 | 3.0 | |
| (25% strength solution) | | |
| Water | 74.6 | |
| 1,2-Propylene glycol | 1.5 | propylene glycol |
| Germall II | 0.1 | imidazolidinylurea |

### Preparation:

The oil and water phases are weighed in separately and homogenized at a temperature of about 80° C. The water phase is then slowly stirred into the oil phase and slowly cooled to room temperature with stirring.

The example can be repeated with the crosslinked polyurethanes from examples 2, 3, 4, 5, 6, 7, 8, 9, 10, 11, 12, 13 and 14. In every case, a standard O/W cream with good properties is obtained.

### Example 12

### Liquid Make-Up

A

| | |
|---|---|
| 1.70 | glyceryl stearate |
| 1.70 | cetyl alcohol |
| 1.70 | Ceteareth-6 |
| 1.70 | Ceteareth-25 |
| 5.20 | caprylic/capric triglyceride |
| 5.20 | mineral oil |

B

| | |
|---|---|
| q.s. | preservative |
| 4.30 | propylene glycol |
| 12.50 | polymer 1 (20% strength aqueous solution) |
| 49.50 | dist. water |

C

| | |
|---|---|
| q.s. | perfume oil |

D

| | |
|---|---|
| 2.00 | iron oxide |
| 12.00 | titanium dioxide |

### Preparation:

Heat phase A and phase B to 80° C. separately from one another. Then mix phase B into phase A using a stirrer. Allow everything to cool to 40° C. and add phase C and phase D. Homogenize a number of times.

The examples can be repeated with the crosslinked polyurethanes of examples 2, 3, 4, 5, 6, 7, 8, 9, 10, 11, 12, 13 and 14. In every case, a liquid make-up with good properties is obtained.

### Example 13

### Oil-Free Make-Up

A

| | |
|---|---|
| 0.35 | veegum |
| 5.00 | butylene glycol |
| 0.15 | xanthan gum |

B

| | |
|---|---|
| 34.0 | dist. water |
| q.s. | preservative |
| 0.2 | Polysorbate-20 |
| 1.6 | tetrahydroxypropylethylenediamine |

C

| | |
|---|---|
| 1.0 | silicon dioxide |
| 2.0 | Nylon-12 |
| 4.15 | mica |
| 6.0 | titanium dioxide |
| 1.85 | iron oxide |

D

| | |
|---|---|
| 4.0 | stearic acid |
| 1.5 | glyceryl stearate |
| 7.0 | benzyl laurate |
| 5.0 | isoeicosane |
| q.s. | preservative |

E

| | |
|---|---|
| 0.5 | panthenol |
| 0.1 | imidazolidinylurea |
| 25.0 | polymer 1 (20% strength aqueous solution) |

### Preparation:

Wet phase A with butylene glycol, add to phase B and mix well. Heat phase AB to 75° C. Pulverize phase C feed materials, add to phase AB and homogenize well. Mix feed materials of phase D, heat to 80° C. and add to phase ABC. Mix for some time until everything is homogenous. Transfer everything to a vessel with propeller mixer. Mix feed materials of phase E, add to phase ABCD and mix well.

The example can be repeated with the crosslinked polyurethanes from examples 2, 3, 4, 5, 6, 7, 8, 9, 10, 11, 12, 13 and 14. In every case, an oil-free make-up with good properties is obtained.

### Example 14

### Shimmering Gel

A

| | |
|---|---|
| 32.6 | dist. water |
| 0.1 | disodium EDTA |
| 25.0 | Natrosol (4% strength aqueous solution) |
| 0.3 | preservative |

B

| | |
|---|---|
| 0.5 | dist. water |
| 0.5 | triethanolamine |

C

| | |
|---|---|
| 9.0 | polymer 1 (25% strength aqueous solution) |
| 1.0 | polyquaternium-46 (20% strength aqueous solution) |
| 5.0 | iron oxide |

D

| | |
|---|---|
| 15.0 | dist. water |
| 1.0 | D-Panthenol 50 P (panthenol and propylene glycol) |

### Preparation:

Using a propeller mixer, thoroughly mix the feed materials of phase A in the order given. Then add phase B to phase A. Stir slowly until everything is homogenous. Homogenize phase C thoroughly until the pigments are well distributed. Add phase C and phase D to phase AB and mix well.

The example can be repeated with the crosslinked polyurethanes of examples 2, 3, 4, 5, 6, 7, 8, 9, 10, 11, 12, 13 and 14. In every case, a shimmering gel with good properties is obtained.

### Example 15

### Sunscreen Gel

Phase A

| | |
|---|---|
| 1.00 | hydrogenated castor oil PEG-40 |
| 8.00 | octyl methoxycinnamate (Uvinul MC 80TM from BASF) |
| 5.00 | octocrylene (Uvinul N 539 TM from BASF) |
| 0.80 | octyltriazone (Uvinul T 150 TM from BASF) |
| 2.00 | butylmethoxydibenzoylmethane (Uvinul BMBM |
| | TM from BASF) |
| 2.00 | tocopheryl acetate |
| q.s. | perfume oil |

Phase B

| | |
|---|---|
| 12.50 | polymer 1 (20% strength aqueous solution) |
| 0.30 | acrylate/C₁₀₋₃₀ alkyl acrylate copolymer |
| 0.20 | carbomer |
| 5.00 | glycerol |
| 0.20 | disodium EDTA |
| q.s. | preservative |
| 62.80 | dist. water |

Phase C

| | |
|---|---|
| 0.20 | sodium hydroxide |

### Preparation:

Mix the components of phase A. Allow phase B to swell and stir into phase A with homogenization. Neutralize with phase C and homogenize again.

The example can be repeated with the crosslinked polyurethanes of examples 2, 3, 4, 5, 6, 7, 8, 9, 10, 11, 12, 13 and 14. In every case, a sunscreen gel with good properties is obtained.

### Example 16

### Sunscreen Emulsion Containing TiO₂ and ZnO₂

Phase A

| | |
|---|---|
| 6.00 | hydrogenated castor oil PEG-7 |
| 2.00 | PEG-45/dodecyl glycol copolymer |
| 3.00 | isopropyl myristate |
| 8.00 | jojoba oil (Buxus chinensis) |
| 4.00 | octyl methoxycinnamate (Uvinul MC 80) |
| 2.00 | 4-methylbenzylidenecamphor (Uvinul MBC 95) |
| 3.00 | titanium dioxide, dimethicone |
| 1.00 | dimethicone |
| 5.00 | zinc oxide, dimethicone |

Phase B

| | |
|---|---|
| 10.00 | polymer 1 (20% strength aqueous solution) |
| 0.20 | disodium EDTA |
| 5.00 | glycerol |
| q.s. | preservative |
| 50.80 | dist. water |

Phase C

| | |
|---|---|
| q.s. | perfume oil |

### Preparation:

Heat phases A and B separately to about 85° C. Stir phase B into phase A and homogenize. Cool to about 40° C., add phase C and homogenize briefly again.

The example can be repeated with the crosslinked polyurethanes of examples 2, 3, 4, 5, 6, 7, 8, 9, 10, 11, 12, 13 and 14. In every case, a sunscreen emulsion containing TiO₂ and ZnO₂ with good properties is obtained.

### Example 17

### Sunscreen Lotion

Phase A

| | |
|---|---|
| 6.00 | octyl methoxycinnamate (Uvinul MC 80 TM from BASF) |
| 2.50 | 4-methylbenzylidenecamphor (Uvinul MBC 95 TM from BASF) |
| 1.00 | octyltriazone (Uvinul T 150 TM from BASF) |
| 2.00 | butylmethoxydibenzoylmethane (Uvinul BMBM TM from |
| | BASF) |
| 2.00 | PVP/hexadecene copolymer |
| 5.00 | PPG-3 myristyl ether |
| 0.50 | dimethicone |
| 0.10 | BHT, ascorbyl palmitate, citric acid, glyceryl stearate, |
| | propylene glycol |
| 2.00 | cetyl alcohol |
| 2.00 | potassium cetyl phosphate |

Phase B

| | |
|---|---|
| 2.50 | polymer 1 (20% strength aqueous solution) |
| 5.00 | propylene glycol |
| 0.20 | disodium EDTA |
| q.s. | preservative |
| 63.92 | dist. water |

Phase C

| | |
|---|---|
| 5.00 | mineral oil |
| 0.20 | carbomer |

Phase D

| | |
|---|---|
| 0.08 | sodium hydroxide |

Phase E

| | |
|---|---|
| q.s. | perfume oil |

### Preparation:

Heat phases A and B separately to about 80° C. Stir phase B into phase A with homogenization, briefly after-homogenize. Prepare a slurry from phase C, stir into phase AB, neutralize with phase D and after-homogenize. Cool to about 40° C., add phase E, homogenize again.

The example can be repeated with the crosslinked polyurethanes of examples 2, 3, 4, 5, 6, 7, 8, 9, 10, 11, 12, 13 and 14. In every case, a sunscreen lotion with good properties is obtained.

### Example 18

### Peelable Face Mask

Phase A

| | |
|---|---|
| 57.10 | dist. water |
| 6.00 | polyvinyl alcohol |
| 5.00 | propylene glycol |

Phase B

| | |
|---|---|
| 20.00 | alcohol |
| 4.00 | PEG-32 |
| q.s | perfume oil |

Phase C

| | |
|---|---|
| 5.00 | Polyquaternium-44 |
| 2.70 | polymer 1 (20% strength aqueous solution) |
| 0.20 | allantoin |

### Preparation:

Heat phase A to at least 90° C. and stir until dissolved. Dissolve phase B at 50° C. and stir into phase A. At about 35° C., compensate for the loss of ethanol. Add phase C and stir in.

The example can be repeated with the crosslinked polyurethanes of examples 2, 3, 4, 5, 6, 7, 8, 9, 10, 11, 12, 13 and 14. In every case, a peelable face mask with good properties is obtained.

### Example 19

### Face Mask

Phase A

| | |
|---|---|
| 3.00 | Ceteareth-6 |
| 1.50 | Ceteareth-25 |
| 5.00 | cetearyl alcohol |
| 6.00 | cetearyl octanoate |
| 6.00 | mineral oil |
| 0.20 | bisabolol |
| 3.00 | glyceryl stearate |

Phase B

| | |
|---|---|
| 2.00 | propylene glycol |
| 5.00 | panthenol |
| 14.00 | polymer 1 (20% strength aqueous solution) |
| q.s. | preservative |
| 53.80 | dist. water |

Phase C

| | |
|---|---|
| q.s. | perfume oil |
| 0.50 | tocopheryl acetate |

### Preparation:

Heat phase A and B separately to about 80° C. Stir phase B into phase A with homogenization, briefly after-homogenize. Cool to about 40° C., add phase C, homogenize again.

The example can be repeated with the crosslinked polyurethanes of examples 2, 3, 4, 5, 6, 7, 8, 9, 10, 11, 12, 13 and 14. In every case, a face mask with good properties is obtained.

### Example 20

### Body Lotion Foam

Phase A

| | |
|---|---|
| 1.50 | Ceteareth-25 |
| 1.50 | Ceteareth-6 |
| 4.00 | cetearyl alcohol |
| 10.00 | cetearyl octanoate |
| 1.00 | dimethicone |

Phase B

| | |
|---|---|
| 3.00 | polymer 1 (20% strength aqueous solution) |
| 2.00 | panthenol |
| 2.50 | propylene glycol |
| q.s. | preservative |
| 74.50 | dist. water |

Phase C

| | |
|---|---|
| q.s. | perfume oil |

### Preparation:

Heat phases A and B separately to about 80° C. Stir phase B into phase A and homogenize. Cool to about 40° C., add phase C and homogenize briefly again. Bottling: 90% active ingredient and 10% propane/butane at 3.5 bar (20° C.).

The example can be repeated with the crosslinked polyurethanes of examples 2, 3, 4, 5, 6, 7, 8, 9, 10, 11, 12, 13 and 14. In every case, a body lotion foam with good properties is obtained.

### Example 21

### Face Tonic for Dry and Sensitive Skin

Phase A

| | |
|---|---|
| 2.50 | hydrogenated castor oil PEG-40 |
| q.s. | perfume oil |
| 0.40 | bisabolol |

Phase B

| | |
|---|---|
| 3.00 | glycerol |
| 1.00 | hydroxyethylcetyldimonium phosphate |
| 5.00 | witch hazel (Hamamelis virginiana) distillate |
| 0.50 | panthenol |
| 0.50 | polymer 1 (25% strength aqueous solution) |
| q.s. | preservative |
| 87.60 | dist. water |

### Preparation:

Dissolve phase A until clear. Stir phase B into phase A.

The example can be repeated with the crosslinked polyurethanes of examples 2, 3, 4, 5, 6, 7, 8, 9, 10, 11, 12, 13 and 14. In every case, a face tonic for dry and sensitive skin with good properties is obtained.

### Example 22

### Face Wash Paste with Peeling Effect

Phase A

| | |
|---|---|
| 58.00 | dist. water |
| 15.00 | polymer 1 (20% strength aqueous solution) |
| 1.50 | carbomer |
| q.s. | preservative |

Phase B

| | |
|---|---|
| q.s. | perfume oil |
| 7.00 | potassium cocoyl hydrolyzed protein |
| 4.00 | cocamidopropylbetaine |

Phase C

| | |
|---|---|
| 1.50 | triethanolamine |

Phase D

| | |
|---|---|
| 13.00 | polyethylene (Luwax ATM from BASF) |

### Preparation:

Allow phase A to swell. Dissolve phase B until clear. Stir phase B into phase A.

Neutralize with phase C. Then stir in phase D.

The example can be repeated with the crosslinked polyurethanes of examples 2, 3, 4, 5, 6, 7, 8, 9, 10, 11, 12, 13 and 14. In every case, a face wash paste with peeling effect with good properties is obtained.

### Example 23

### Face Soap

Phase A

| | |
|---|---|
| 25.0 | potassium cocoate |
| 20.0 | disodium cocoamphodiacetate |
| 2.0 | lauramide DEA |
| 1.0 | glycol stearate |
| 2.0 | polymer 1 (25% strength aqueous solution) |
| 50.0 | dist. water |
| q.s. | citric acid |

Phase B

| | |
|---|---|
| q.s. | preservative |
| q.s. | perfume oil |

### Preparation:

Heat phase A to 70° C. with stirring until everything is homogeneous, adjust pH to 7.0-7.5 with citric acid, allow everything to cool to 50° C. and add phase B.

The example can be repeated with the crosslinked polyurethanes of examples 2, 3, 4, 5, 6, 7, 8, 9, 10, 11, 12, 13 and 14. In every case, a face soap with good properties is obtained.

### Example 24

### Face Cleansing Milk, O/W Type

Phase A

| | |
|---|---|
| 1.50 | Ceteareth-6 |
| 1.50 | Ceteareth-25 |
| 2.00 | glyceryl stearate |
| 2.00 | cetyl alcohol |
| 10.00 | mineral oil |

Phase B

| | |
|---|---|
| 5.00 | propylene glycol |
| q.s. | preservative |
| 5.0 | polymer 1 (20% strength aqueous solution) |
| 62.30 | dist. water |

Phase C

| | |
|---|---|
| 0.20 | carbomer |
| 10.00 | cetearyl octanoate |

Phase D

| | |
|---|---|
| 0.40 | tetrahydroxypropylethylenediamine |

Phase E

| | |
|---|---|
| q.s. | perfume oil |
| 0.10 | bisabolol |

### Preparation:

Heat phases A and B separately to about 80° C. Stir phase B into phase A with homogenization, briefly after-homogenize. Prepare a slurry from phase C, stir into phase AB, neutralize with phase D and after-homogenize. Cool to about 40° C., add phase E, homogenize again.

The example can be repeated with the crosslinked polyurethanes of examples 2, 3, 4, 5, 6, 7, 8, 9, 10, 11, 12, 13 and 14. In every case, a face cleansing milk, O/W type with good properties is obtained.

### Example 25

### Peeling Cream, O/W Type

Phase A

| | |
|---|---|
| 3.00 | Ceteareth-6 |
| 1.50 | Ceteareth-25 |
| 3.00 | glyceryl stearate |
| 5.00 | cetearyl alcohol, sodium cetearyl sulfate |
| 6.00 | cetearyl octanoate |
| 6.00 | mineral oil |
| 0.20 | bisabolol |

Phase B

| | |
|---|---|
| 2.00 | propylene glycol |
| 0.10 | disodium EDTA |
| 3.00 | polymer 1 (25% strength aqueous solution) |
| q.s. | preservative |
| 59.70 | dist. water |

Phase C

| | |
|---|---|
| 0.50 | tocopheryl acetate |
| q.s. | perfume oil |

Phase D

| | |
|---|---|
| 10.00 | polyethylene |

### Preparation:

Heat phases A and B separately to about 80° C. Stir phase B into phase A and homogenize. Cool to about 40° C., add phase C and briefly homogenize again. Then stir phase D in.

The example can be repeated with the crosslinked polyurethanes of examples 2, 3, 4, 5, 6, 7, 8, 9, 10, 11, 12, 13 and 14. In every case, a peeling cream, O/W type with good properties is obtained.

### Example 26

### Shaving Foam

| | |
|---|---|
| 6.00 | Ceteareth-25 |
| 5.00 | Poloxamer 407 |
| 52.00 | dist. water |
| 1.00 | triethanolamine |
| 5.00 | propylene glycol |
| 1.00 | lanolin oil PEG-75 |
| 5.00 | polymer 1 according to the invention |
| | (20% strength aqueous solution) |
| q.s. | preservative |
| q.s. | perfume oil |
| 25.00 | sodium laureth sulfate |

### Preparation:

Weigh everything together, then stir until dissolved. Bottling: 90 parts of active substance and 10 parts of 25:75 propane/butane mixture.

The example can be repeated with the crosslinked polyurethanes of examples 2, 3, 4, 5, 6, 7, 8, 9, 10, 11, 12, 13 and 14. In every case, a shaving foam with good properties is obtained.

### Example 27

### Aftershave Balsam

Phase A

| | |
|---|---|
| 0.25 | acrylate/C₁₀₋₃₀ alkyl acrylate copolymer |
| 1.50 | tocopheryl acetate |
| 0.20 | bisabolol |
| 10.00 | caprylic/capric triglyceride |
| q.s. | perfume oil |
| 1.00 | hydrogenated castor oil PEG-40 |

Phase B

| | |
|---|---|
| 1.00 | panthenol |
| 15.00 | alcohol |
| 5.00 | glycerol |
| 0.05 | hydroxyethylcellulose |
| 1.92 | polymer 1 (25% aqueous solution) |
| 64.00 | dist. water |

Phase C

| | |
|---|---|
| 0.08 | sodium hydroxide |

### Preparation:

Mix the components of phase A. Stir phase B into phase A with homogenization, briefly after-homogenize. Neutralize with phase C and homogenize again.

The example can be repeated with the crosslinked polyurethanes of examples 2, 3, 4, 5, 6, 7, 8, 9, 10, 11, 12, 13 and 14. In all cases, an aftershave balsam with good properties is obtained.

### Example 28

### Bodycare Cream

Phase A

| | |
|---|---|
| 2.00 | Ceteareth-6 |
| 2.00 | Ceteareth-25 |
| 2.00 | cetearyl alcohol |
| 3.00 | glyceryl stearate SE |
| 5.00 | mineral oil |
| 4.00 | jojoba oil (Buxus chinensis) |
| 3.00 | cetearyl octanoate |
| 1.00 | dimethicone |
| 3.00 | mineral oil, lanolin alcohol |

Phase B

| | |
|---|---|
| 5.00 | propylene glycol |
| 0.50 | veegum |
| 1.00 | panthenol |
| 8.50 | polymer 1 (20% strength aqueous solution) |
| 6.00 | Polyquaternium-44 (10% strength aqueous |
| | solution) |
| q.s. | preservative |
| 54.00 | dist. water |

Phase C

| | |
|---|---|
| q.s. | perfume oil |

### Preparation:

Heat phases A and B separately to about 80° C. Homogenize phase B. Stir phase B with homogenization into phase A, briefly after-homogenize. Cool to about 40° C., add phase C and briefly homogenize again.

The example can be repeated with the crosslinked polyurethanes of examples 2, 3, 4, 5, 6, 7, 8, 9, 10, 11, 12, 13 and 14. In every case, a bodycare cream with good properties is obtained.

### Example 29

### Toothpaste

Phase A

| | |
|---|---|
| 34.79 | dist. water |
| 3.00 | polymer 1 (25% strength aqueous solution) |
| 0.30 | preservative |
| 20.00 | glycerol |
| 0.76 | sodium monofluorophosphate |

Phase B

| | |
|---|---|
| 1.20 | sodium carboxymethylcellulose |

Phase C

| | |
|---|---|
| 0.80 | aroma oil |
| 0.06 | saccharin |
| 0.10 | preservative |
| 0.05 | bisabolol |
| 1.00 | panthenol |
| 0.50 | tocopheryl acetate |
| 2.80 | silicon dioxide |
| 1.00 | sodium lauryl sulfate |
| 7.90 | dicalcium phosphate, anhydrous |
| 25.29 | dicalcium phosphate dihydrate |
| 0.45 | titanium dioxide |

### Preparation:

Dissolve phase A. Sprinkle phase B into phase A and dissolve. Add phase C and leave to stir at RT for about 45 min under reduced pressure.

The example can be repeated with the crosslinked polyurethanes of examples 2, 3, 4, 5, 6, 7, 8, 9, 10, 11, 12, 13 and 14. In every case, a toothpaste with good properties is obtained.

### Example 30

### Mouthwash

Phase A

| | |
|---|---|
| 2.00 | aroma oil |
| 4.00 | hydrogenated castor oil PEG-40 |
| 1.00 | bisabolol |
| 30.00 | alcohol |

Phase B

| | |
|---|---|
| 0.20 | saccharin |
| 5.00 | glycerol |
| q.s. | preservative |
| 5.00 | Poloxamer 407 |
| 2.5 | polymer 1 (20% strength aqueous solution) |
| 50.30 | dist. water |

### Preparation:

Dissolve phase A and phase B separately until clear. Stir phase B into phase A

The example can be repeated with the crosslinked polyurethanes of examples 2, 3, 4, 5, 6, 7, 8, 9, 10, 11, 12, 13 and 14. In every case, a mouthwash with good properties is obtained.

### Example 31

### Prosthesis Adhesive

Phase A

| | |
|---|---|
| 0.20 | bisabolol |
| 1.00 | betacarotene |
| q.s. | aroma oil |
| 20.00 | cetearyl octanoate |
| 5.00 | silicon dioxide |
| 33.80 | mineral oil |

Phase B

| | |
|---|---|
| 5.00 | polymer 1 (20% strength aqueous solution) |
| 35.00 | PVP (20% strength solution in water) |

### Preparation:

Mix phase A thoroughly. Stir phase B into phase A.

The example can be repeated with the crosslinked polyurethanes of examples 2, 3, 4, 5, 6, 7, 8, 9, 10, 11, 12, 13 and 14. In every case, a prosthesis adhesive with good properties is obtained.

### Example 32

### Skincare Cream, O/W Type

Phase A

| | |
|---|---|
| 8.00 | cetearyl alcohol |
| 2.00 | Ceteareth-6 |
| 2.00 | Ceteareth-25 |
| 10.00 | mineral oil |
| 5.00 | cetearyl octanoate |
| 5.00 | dimethicone |

Phase B

| | |
|---|---|
| 3.00 | polymer 1 (25% strength aqueous solution) |
| 2.00 | panthenol, propylene glycol |
| q.s. | preservative |
| 63.00 | dist. water |

Phase C

| | |
|---|---|
| q.s. | perfume oil |

### Preparation:

Heat phase A and phase B separately to about 80° C. Stir phase B into phase A with homogenization, briefly after-homogenize. Cool to about 40° C., add phase C, homogenize again.

The example can be repeated with the crosslinked polyurethanes of examples 2, 3, 4, 5, 6, 7, 8, 9, 10, 11, 12, 13 and 14. In every case, a skincare cream, O/W type with good properties is obtained.

### Example 33

### Skincare Cream, O/W Type

Phase A

| | |
|---|---|
| 6.00 | hydrogenated castor oil PEG-7 |
| 8.00 | cetearyl octanoate |
| 5.00 | isopropyl myristate |
| 15.00 | mineral oil |
| 2.00 | PEG-45/dodecyl glycol copolymer |
| 0.50 | magnesium stearate |
| 0.50 | aluminum stearate |

Phase B

| | |
|---|---|
| 3.00 | glycerol |
| 3.30 | polymer 1 (20% strength aqueous solution) |
| 0.70 | magnesium sulfate |
| 2.00 | panthenol |
| q.s. | preservative |
| 48.00 | dist. water |

Phase C

| | |
|---|---|
| 1.00 | tocopherol |
| 5.00 | tocopheryl acetate |
| q.s. | perfume oil |

### Preparation:

Heat phases A and B separately to about 80° C. Stir phase B into phase A and homogenize. Cool to about 40° C., add phase C and birefly homogenize again.

The example can be repeated with the crosslinked polyurethanes of examples 2, 3, 4, 5, 6, 7, 8, 9, 10, 11, 12, 13 and 14. In every case, a skincare cream, W/O type with good properties is obtained.

### Example 34

### Lipcare Cream

Phase A

| | |
|---|---|
| 10.00 | cetearyl octanoate |
| 5.00 | polybutene |

Phase B

| | |
|---|---|
| 0.10 | carbomer |

Phase C

| | |
|---|---|
| 2.00 | Ceteareth-6 |
| 2.00 | Ceteareth-25 |
| 2.00 | glyceryl stearate |
| 2.00 | cetyl alcohol |
| 1.00 | dimethicone |
| 1.00 | Benzophenone-3 |
| 0.20 | bisabolol |
| 6.00 | mineral oil |

Phase D

| | |
|---|---|
| 8.00 | polymer 1 (20% strength aqueous solution) |
| 3.00 | panthenol |
| 3.00 | propylene glycol |
| q.s. | preservative |
| 54.00 | dist. water |

Phase E

| | |
|---|---|
| 0.10 | triethanolamine |

Phase F

| | |
|---|---|
| 0.50 | tocopheryl acetate |
| 0.10 | tocopherol |
| q.s. | perfume oil |

### Preparation:

Dissolve phase A until clear. Add phase B and homogenize. Add phase C and melt at 80° C. Heat phase D to 80° C. Add phase D to phase ABC and homogenize. Cool to about 40° C., add phase E and phase F, homogenize again.

The example can be repeated with the crosslinked polyurethanes of examples 2, 3, 4, 5, 6, 7, 8, 9, 10, 11, 12, 13 and 14. In every case, a lipcare cream with good properties is obtained.

### Example 35

### Shower Gel

| | |
|---|---|
| 50.00 | sodium laureth sulfate, magnesium |
| | laureth sulfate, Sodium Laureth-8 |
| | Sulfate, Magnesium Laureth-8 |
| 1.00 | cocoamide DEA |
| 4.00 | polymer 1 (25% strength aqueous solution) |
| 2.00 | sodium laureth sulfate, glycol distearate, |
| | cocamide MEA, Laureth-10 |
| q.s. | preservative |
| q.s. | perfume oil |
| 2.00 | sodium chloride |
| 41.00 | dist. water |

### Preparation:

Weigh all of the components in together and stir until dissolved.

The example can be repeated with the crosslinked polyurethanes of examples 2, 3, 4, 5, 6, 7, 8, 9, 10, 11, 12, 13 and 14. In every case, a shower gel with good properties is obtained.

### Example 36

### Shower Gel

| | |
|---|---|
| 30.00 | sodium laureth sulfate |
| 6.00 | sodium cocoamphodiacetate |
| 6.00 | cocamidopropylbetaine |
| 3.00 | sodium laureth sulfate, glycol distearate, |
| | cocamide MEA, Laureth-10 |
| 7.70 | Polyquaternium-44 |
| 1.50 | polymer 1 (25% strength aqueous solution) |
| 1.00 | panthenol |
| q.s. | preservative |
| q.s. | perfume oil |
| q.s. | citric acid |
| 0.50 | sodium chloride |
| 44.30 | dist. water |

### Preparation:

Weigh in the components of phase A and dissolve. Adjust the pH to 6 to 7.

The example can be repeated with the crosslinked polyurethanes of examples 2, 3, 4, 5, 6, 7, 8, 9, 10, 11, 12, 13 and 14 In every case, a shower gel with good properties is obtained.

### Example 37

### Clear Shower Gel

| | |
|---|---|
| 40.00 | sodium laureth sulfate |
| 5.00 | decyl glucoside |
| 5.00 | cocamidopropylbetaine |
| 0.50 | Polyquaternium-10 |
| 11.00 | polymer 1 (20% strength aqueous solution) |
| 1.00 | panthenol |
| q.s. | perfume oil |
| q.s. | preservative |
| q.s. | citric acid |
| 2.00 | sodium chloride |
| 35.50 | dist. water |

### Preparation:

Weigh in the components of phase A and dissolve until clear.

The example can be repeated with the crosslinked polyurethanes of examples 2, 3, 4, 5, 6, 7, 8, 9, 10, 11, 12, 13 and 14. In every case, a clear shower gel with good properties is obtained.

### Example 38

### Shower Bath

A

| | |
|---|---|
| 40.00 | sodium laureth sulfate |
| 5.00 | sodium C₁₂₋₁₅ Pareth-15 sulfonate |
| 5.00 | decyl glucoside |
| q.s. | perfume oil |
| 0.10 | phytantriol |

B

| | |
|---|---|
| 34.80 | dist. water |
| 0.1 | guar hydroxypropyltrimonium chloride |
| 11.00 | polymer 1 (20% strength aqueous solution) |
| 1.00 | panthenol |
| q.s. | preservative |
| 1.00 | Laureth-3 |
| q.s. | citric acid |
| 2.00 | sodium chloride |

### Preparation:

Mix the components of phase A. Add the components of phase B one after the other and mix. Adjust the pH to 6 to 7.

The example can be repeated with the crosslinked polyurethanes of examples 2, 3, 4, 5, 6, 7, 8, 9, 10, 11, 12, 13 and 14. In every case, a shower bath with good properties is obtained.

### Example 39

### Liquid Soap

A

| | |
|---|---|
| 43.26 | dist. water |
| 0.34 | aminomethylpropanol |
| 3.40 | poly(ethyl acrylate/methacrylic acid) |
| | (Luviflex Soft ®, BASF) |

B

| | |
|---|---|
| 40.00 | sodium laureth sulfate |
| 10.00 | cocamidopropylbetaine |
| 1.00 | polymer 1 (20% strength aqueous solution) |
| q.s. | perfume oil |
| q.s. | preservative |
| 2.00 | sodium chloride |

### Preparation:

Weigh in the components of phase A and dissolve until clear. Add the components of phase B one after the other and mix.

The example can be repeated with the crosslinked polyurethanes of examples 2, 3, 4, 5, 6, 7, 8, 9, 10, 11, 12, 13 and 14. In every case, a liquid soap with good properties is obtained.

### Example 40

### Liquid Footbath

A

| | |
|---|---|
| 1.00 | Nonoxynol-14 |
| 0.10 | bisabolol |
| 1.00 | pine oil (Pinus sylvestris) |

B

| | |
|---|---|
| 5.00 | PEG-8 |
| 6.00 | polymer 1 (20% strength aqueous solution) |
| 0.50 | triclosan |
| 30.00 | sodium laureth sulfate |
| 3.00 | Polyquaternium-16 |
| 53.40 | dist. water |
| q.s. | C.I. 19 140 + C.I. 42 051 |

### Preparation:

Solubilize phase A. Mix phase B.

The example can be repeated with the crosslinked polyurethanes of examples 2, 3, 4, 5, 6, 7, 8, 9, 10, 11, 12, 13 and 14. In every case, a liquid footbath with good properties is obtained.

### Example 41

### Freshening Gel

A

| | |
|---|---|
| 0.60 | carbomer |
| 45.40 | dist. water |

B

| | |
|---|---|
| 0.50 | bisabolol |
| 0.50 | farnesol |
| q.s. | perfume oil |
| 5.00 | PEG-40 hydrogenated castor oil |
| 2.50 | polymer 1 (20% strength aqueous solution) |
| 1.00 | tetrahydroxypropylethylenediamine |
| 1.50 | menthol |
| 43.00 | alcohol |
| q.s. | C.I. 74 180, Direct Blue 86 |

### Preparation:

Allow phase A to swell. Dissolve phase B. Stir phase B into phase A.

The example can be repeated with the crosslinked polyurethanes of examples 2, 3, 4, 5, 6, 7, 8, 9, 10, 11, 12, 13 and 14. In every case, a freshing gel with good properties is obtained.

### Example 42

### Roll-on Antiperspirant

A

| | |
|---|---|
| 0.40 | hydroxyethylcellulose |
| 50.00 | dist. water |

B

| | |
|---|---|
| 25.00 | alcohol |
| 0.10 | bisabolol |
| 0.30 | farnesol |
| 2.00 | PEG-40 hydrogenated castor oil |
| q.s. | perfume oil |

C

| | |
|---|---|
| 5.00 | aluminum chlorohydrate |
| 3.00 | propylene glycol |
| 3.00 | dimethicone copolyol |
| 3.00 | Polyquaternium-16 |
| 6.00 | polymer 1 (20% strength aqueous solution) |
| 2.20 | dist. water |

### Preparation:

Allow phase A to swell. Dissolve phase B and C separately. Stir phase A and B into phase C.

The example can be repeated with the crosslinked polyurethanes of examples 2, 3, 4, 5, 6, 7, 8, 9, 10, 11, 12, 13 and 14. In every case, a roll-on antiperspirant with good properties is obtained.

### Example 43

### Transparent Deodorant Stick

| | |
|---|---|
| 5.00 | sodium stearate |
| 0.50 | triclosan |
| 3.00 | Ceteareth-25 |
| 20.00 | glycerol |
| 2.50 | polymer 1 (20% strength aqueous solution) |
| q.s. | perfume oil |
| 60.00 | propylene glycol |
| 0.20 | bisabolol |
| 10.80 | dist. water |

### Preparation:

Weigh phase A together, melt and homogenize. Then pour into the mold.

The example can be repeated with the crosslinked polyurethanes of examples 2, 3, 4, 5, 6, 7, 8, 9, 10, 11, 12, 13 and 14. In every case, a transparent deodorant stick with good properties is obtained.

### Example 44

### Water-Soluble Bath Oil

| | |
|---|---|
| 15.00 | cetearyl octanoate |
| 15.00 | caprylic/capric triglyceride |
| 1.00 | panthenol, propylene glycol |
| 0.10 | bisabolol |
| 2.00 | tocopheryl acetate |
| 2.00 | retinyl palmitate |
| 0.10 | tocopherol |
| 37.00 | PEG-7 glyceryl cocoate |
| 2.00 | polymer 1 (20% strength aqueous solution) |
| 2.20 | dist. water |
| q.s. | perfume oil |
| 23.60 | PEG-40 hydrogenated castor oil |

### Preparation:

Mix and stir until everything has dissolved to give a clear solution.

The example can be repeated with the crosslinked polyurethanes of examples 2, 3, 4, 5, 6, 7, 8, 9, 10, 11, 12, 13 and 14. In every case, a water-soluble bath oil with good properties is obtained.

### Example 45

### Daycare Aerosol

A

| | |
|---|---|
| 4.00 | ethylhexyl methoxycinnamate |
| 1.50 | octocrylene |
| 9.00 | caprylic/capric triglyceride |
| 5.00 | Simmondsia chinensis (jojoba) seed oil |
| 1.50 | cyclomethicone |
| 3.00 | hydrogenated cocoglycerides |
| 1.00 | PVP/hexadecene copolymer |
| 1.00 | Ceteareth-6, stearyl alcohol |

B

| | |
|---|---|
| 5.00 | zinc oxide |

C

| | |
|---|---|
| 2.00 | Ceteareth-25 |
| 1.20 | panthenol |
| 0.20 | sodium ascorbyl phosphate |
| 0.30 | imidazolidinylurea |
| 0.10 | disodium EDTA |
| 7.50 | polymer 1 (20% strength aqueous solution) |
| 56.67 | dist. water |

D

| | |
|---|---|
| 0.50 | tocopheryl acetate |
| 0.20 | bisabolol |
| 0.33 | caprylic/capric triglyceride, retinol |
| q.s. | perfume oil |

### Preparation:

Heat phase A to 80° C. Dissolve phase A until clear. Incorporate phase B and homogenize. Add phase C, heat to 80° C., melt and homogenize. Cool to about 40° C. with stirring. Add phase D and briefly homogenize. Bottle 90% active ingredient solution: 10% propane/butane at 3.5 bar (20° C.).

The example can be repeated with the crosslinked polyurethanes of examples 2, 3, 4, 5, 6, 7, 8, 9, 10, 11, 12, 13 and 14. In every case, a daycare aerosol with good properties is obtained.

### Example 46

### Moisturizing Cream

A

| | |
|---|---|
| 3.00 | Vitis vinifera (grape) seed oil |
| 1.00 | cyclopentasiloxane, cyclohexasiloxane |
| 1.50 | cyclomethicone |
| 2.00 | soybean (glycine soya) oil |
| 2.00 | ethylhexyl methoxycinnamate |
| 1.00 | Uvinul A Plus (BASF) |
| 1.00 | hydrogenated lecithin |
| 1.00 | cholesterol |
| 2.00 | PEG-40 hydrogenated castor oil |
| 5.00 | cetearyl octanoate |
| 5.00 | caprylic/capric triglyceride |

B

| | |
|---|---|
| 3.00 | caprylic/capric triglyceride, acrylate |
| | copolymer |

C

| | |
|---|---|
| 3.00 | polymer 1 (20% strength aqueous solution) |
| 0.50 | cocotrimonium methosulfate |
| 2.00 | panthenol, propylene glycol |
| 3.00 | glycerol |
| 0.10 | disodium EDTA |
| 60.30 | dist. water |

D

| | |
|---|---|
| 0.30 | perfume oil |
| 0.30 | DMDM hydantoin |
| 1.00 | tocopheryl acetate |
| 2.00 | tocopherol |

### Preparation:

Heat phase A to 80° C. Stir phase B into phase A. Heat phase C to about 80° C. and stir into phase A+B with homogenization. Cool to about 40° C. with stirring, add phase D and briefly homogenize.

The example can be repeated with the crosslinked polyurethanes of examples 2, 3, 4, 5, 6, 7, 8, 9, 10, 11, 12, 13 and 14. In every case, a moisturizing cream with good properties is obtained.

### Example 47

### Aerosol Hair Foam

A

| | |
|---|---|
| 2.00 | cocotrimonium methosulfate |
| 0.20 | perfume oil |

B

| | |
|---|---|
| 63.90 | dist. water |
| 6.70 | polymer 1 (25% strength aqueous solution) |
| 0.50 | poly(ethyl acrylate/methacrylic acid) (Luviflex Soft ®, BASF) |
| 0.10 | aminomethylpropanol |
| 0.20 | Ceteareth-25 |
| 0.20 | trimethylsilylamodimethicone, Trideceth-10, cetrimonium chloride |
| 0.10 | PEG-25 PABA |
| 0.20 | hydroxyethylcellulose |
| 0.20 | PEG-8 |
| 0.20 | panthenol |
| 15.00 | alcohol |

C

| | |
|---|---|
| 10.00 | propane/butane 3.5 bar (20° C.) |

### Preparation:

Mix phases A and B and bottle with propellant gas.

The example can be repeated with the crosslinked polyurethanes of examples 2, 3, 4, 5, 6, 7, 8, 9, 10, 11, 12, 13 and 14. In every case, an aerosol hair foam with good properties is obtained.

### Example 48

### Pump Mousse

A

| | |
|---|---|
| 2.00 | cocotrimonium methosulfate |
| q.s. | perfume oil |

B

| | |
|---|---|
| 74.30 | dist. water |
| 7.00 | Polyquaternium-46 (10% strength aqueous solution) |
| 15.00 | polymer 1 (20% strength aqueous solution) |
| 0.50 | PEG-8 |
| 1.00 | panthenol |
| q.s. | preservative |
| 0.20 | PEG-25 PABA (ethoxylated p-aminobenzoic acid) |

### Preparation:

Mix the components of phase A. Add the components of phase B one after the other and dissolve until clear.

The example can be repeated with the crosslinked polyurethanes of examples 2, 3, 4, 5, 6, 7, 8, 9, 10, 11, 12, 13 and 14. In every case, a pump mousse with good properties is obtained.

### Example 49

### Aerosol Foam

| | |
|---|---|
| 15.00 | polymer 1 (20% strength aqueous solution) |
| 5.00 | PVP/VA copolymer (40% strength aqueous |
| | solution) |
| 0.50 | hydroxyethylcetyldimonium phosphate |
| 0.20 | Ceteareth-25 |
| 0.40 | perfume oil PC 910.781/cremophore |
| 68.90 | dist. water |
| q.s. | preservative |
| 10.00 | propane/butane 3.5 bar (20° C.) |

### Preparation:

Weigh everything together, stir until dissolved, then bottle.

The example can be repeated with the crosslinked polyurethanes of examples 2, 3, 4, 5, 6, 7, 8, 9, 10, 11, 12, 13 and 14. In every case, an aerosol foam with good properties is obtained.

### Example 50

### Color Styling Mousse

A

| | |
|---|---|
| 2.00 | cocotrimonium methosulfate |
| q.s. | perfume oil |

B

| | |
|---|---|
| 33.50 | polymer 1 (20% strength aqueous solution) |
| 0.50 | acrylate copolymer (Luvimer 100 P ®, |
| | BASF) |
| 0.10 | aminomethylpropanol |
| 0.20 | Ceteareth-25 |
| 0.20 | panthenol |
| 0.20 | hydroxyethylcellulose |
| 10.00 | alcohol |
| 43.17 | dist. water |
| 0.08 | C.I. 12245, Basic Red 76 |
| 0.05 | C.I. 42510, Basic Violet 14 |

C

| | |
|---|---|
| 10.00 | propane/butane 3.5 bar (20° C.) |

### Preparation:

Weigh everything together, stir until dissolved, then bottle. Only suitable for dark blond and brown hair!

The example can be repeated with the crosslinked polyurethanes of examples 2, 3, 4, 5, 6, 7, 8, 9, 10, 11, 12, 13 and 14. In every case, a color styling mousse with good properties is obtained.

### Example 51

### Pump Hair Foam

A

| | |
|---|---|
| 1.50 | cocotrimonium methosulfate |
| q.s. | perfume oil |

B

| | |
|---|---|
| 10.00 | polymer 1 (20% strength aqueous solution) |
| 86.04 | dist. water |

C

| | |
|---|---|
| 0.46 | aminomethylpropanol |
| 4.00 | PEG/PPG-25/25 dimethicone/acrylate copolymer |
| q.s. | preservative |

### Preparation:

Mix phase A. Stir phase B into phase A. Add phase C and stir until dissolved.

The example can be repeated with the crosslinked polyurethanes of examples 2, 3, 4, 5, 6, 7, 8, 9, 10, 11, 12, 13 and 14. In every case, a pump hair foam with good properties is obtained.

### Example 52

### Aqua Wax

| | |
|---|---|
| 50.00 | polymer 1 (20% strength aqueous solution) |
| q.s. | perfume oil |
| q.s. | hydrogenated castor oil PEG-40 |
| 0.10 | diethyl phthalate |
| 0.10 | cetearyl ethylhexanoate |
| 0.10 | PEG-7 glyceryl cocoate |
| 0.10 | preservative |
| 47.70 | dist. water |
| 2.00 | caprylic/capric triglyceride, acrylate |
| | copolymer |

### Preparation:

Mix everything and homogenize. After-stir for 15 minutes.

The example can be repeated with the crosslinked polyurethanes of examples 2, 3, 4, 5, 6, 7, 8, 9, 10, 11, 12, 13 and 14. In every case, an aqua wax with good properties is obtained.

### Example 53

### Rinse-Off Conditioner and Repair Treatment

A

| | |
|---|---|
| 0.20 | cetearyl octanoate |
| 0.10 | phytantriol |
| 2.00 | hydrogenated castor oil PEG-40 |

B

| | |
|---|---|
| q.s. | perfume oil |
| 2.00 | cocotrimonium methosulfate |

C

| | |
|---|---|
| 77.70 | dist. water |

D

| | |
|---|---|
| 2.00 | Polyquaternium-16 (20% strength aqueous solution) |
| 5.00 | polymer 1 (20% strength aqueous solution) |
| 1.00 | dimethicone copolyol |
| q.s. | preservative |
| 10.00 | alcohol |
| q.s. | citric acid |

### Preparation:

Mix phases A and B separately. Stir phase C into phase B.

The example can be repeated with the crosslinked polyurethanes of examples 2, 3, 4, 5, 6, 7, 8, 9, 10, 11, 12, 13 and 14. In every case, a rinse-off conditioner and repair treatment with good properties is obtained.

### Example 54

### Hair Treatment

A

| | |
|---|---|
| 2.00 | Ceteareth-6, stearyl alcohol |
| 1.00 | Ceteareth-25 |
| 6.00 | cetearyl alcohol |
| 6.00 | cetearyl octanoate |
| 0.30 | phytantriol |

B

| | |
|---|---|
| 5.00 | polymer 1 (20% strength aqueous solution) |
| 0.70 | guar hydroxypropyltrimonium chloride |
| 5.00 | propylene glycol |
| 2.00 | panthenol |
| 0.30 | imidazolidinylurea |
| 69.00 | dist. water |

C

| | |
|---|---|
| 2.00 | Cosi Silk Soluble |
| 0.20 | perfume |
| 0.50 | phenoxyethanol |

### Preparation:

Heat phases A and B separately to about 80° C. Homogenize phase B.

The example can be repeated with the crosslinked polyurethanes of examples 2, 3, 4, 5, 6, 7, 8, 9, 10, 11, 12, 13 and 14. In every case, a hair treatment with good properties is obtained.

### Example 55

### Hair Cocktail

A

| | |
|---|---|
| 0.40 | acrylates/C₁₀₋₃₀ alkyl acrylate crosspolymer |
| 2.00 | dimethicone |
| 3.00 | cyclomethicone, dimethiconol |
| 2.00 | phenyltrimethicone |
| 2.00 | amodimethicone, cetrimonium chloride, Trideceth-10 |
| 0.50 | dimethicone copolyol |
| 1.00 | macadamia nut oil (Ternifolia) |
| 0.50 | tocopheryl acetate |
| 1.00 | PEG-40 hydrogenated castor oil |
| q.s. | perfume oil |

B

| | |
|---|---|
| 81.64 | dist. water |
| 1.50 | polymer 1 (20% strength aqueous solution) |
| 0.46 | aminomethylpropanol |
| 4.00 | PEG/PPG-25/25 dimethicone/acrylate copolymer |

### Preparation:

Mix the components of phase A. Dissolve phase B. Stir phase B into phase A with homogenization.

The example can be repeated with the crosslinked polyurethanes of examples 2, 3, 4, 5, 6, 7, 8, 9, 10, 11, 12, 13 and 14. In every case, a hair cocktail with good properties is obtained.

### Example 56

### Permanent Wave

### Waving Solution

A

| | |
|---|---|
| 68.95 | dist. water |
| 0.20 | cocamidopropylbetaine |
| 0.20 | Polysorbate 20 |
| 6.25 | polymer 1 according to the invention |
| | (20% strength aqueous solution) |
| 0.20 | disodium EDTA |
| 0.20 | hydroxyethylcellulose |

B

| | |
|---|---|
| 8.00 | thioglycolic acid |

C

| | |
|---|---|
| 11.00 | ammonium hydroxide |

D

| | |
|---|---|
| 5.00 | ammonium carbonate |

### Preparation:

Weigh in the components of phase A and dissolve until clear. Stir phase B into phase A.

The example can be repeated with the crosslinked polyurethanes of examples 2, 3, 4, 5, 6, 7, 8, 9, 10, 11, 12, 13 and 14. In every case, a waving solution with good properties is obtained.

### Example 57

### Neutralizer

A

| | |
|---|---|
| 1.00 | PEG-40 hydrogenated castor oil |
| 0.20 | perfume oil |
| 83.60 | dist. water |

B

| | |
|---|---|
| 0.20 | cocamidopropylbetaine |
| 0.20 | Ceteareth-25 |
| 12.50 | polymer 1 (20% strength aqueous solution) |
| q.s. | preservative |

C

| | |
|---|---|
| 2.30 | hydrogen peroxide |

D

| | |
|---|---|
| q.s. | phosphoric acid |

### Preparation:

Solubilize phase A. Add the components of phase B one after the other and dissolve until clear.

The example can be repeated with the crosslinked polyurethanes of examples 2, 3, 4, 5, 6, 7, 8, 9, 10, 11, 12, 13 and 14. In every case, a neutralizer with good properties is obtained.

### Example 58

### Dark Brown Permanent Hair Color

### Oxidation Hair Color

A

| | |
|---|---|
| 46.90 | dist. water |
| 0.20 | sodium sulfite |
| 0.05 | disodium EDTA |
| 0.20 | p-phenylenediamine |
| 0.30 | resorcinol |
| 0.20 | 4-amino-2-hydroxytoluene |
| 0.10 | m-aminophenol |
| 1.50 | oleyl alcohol |
| 4.50 | propylene glycol |
| 2.30 | sodium C₁₂₋₁₅ Pareth-15 sulfonate |
| 20.00 | oleic acid |

B

| | |
|---|---|
| 5.00 | polymer 1 (20% strength aqueous solution) |
| 13.70 | ammonium hydroxide |
| 6.00 | i-propanol |
| q.s. | perfume |

### Preparation:

Solubilize phase A. Add the components of phase B one after the other and mix.

The example can be repeated with the crosslinked polyurethanes of examples 2, 3, 4, 5, 6, 7, 8, 9, 10, 11, 12, 13 and 14. In every case, a dark brown permanent hair color (oxidation hair color) with good properties is obtained.

### Example 59

### Developer Emulsion

### pH 3-4

| | |
|---|---|
| 3.00 | hexadecyl alcohol |
| 10.00 | polymer 1 (20% strength aqueous solution) |
| 1.00 | Ceteareth-20 |
| 1.00 | sodium C₁₂₋₁₅ Pareth-15 sulfonate |
| 6.00 | hydrogen peroxide |
| 0.50 | phosphoric acid |
| 0.01 | acetanilide |
| 78.49 | dist. water |

### Preparation:

Add the components one after the other and mix.

The example can be repeated with the crosslinked polyurethanes of examples 2, 3, 4, 5, 6, 7, 8, 9, 10, 11, 12, 13 and 14. In every case, a developer emulsion (pH 3-4) with good properties is obtained.

### Example 60

### Pale Brown Semipermanent Hair Color

| | |
|---|---|
| 10.00 | cocodiethanolamide |
| 4.00 | sodium dodecylbenzyl sulfonate, 50% strength |
| 5.00 | polymer 1 (20% strength aqueous solution) |
| 6.00 | C₉₋₁₁ Pareth-3 |
| 2.50 | sodium lauryl sulfate |
| 0.40 | 2-nitro-p-phenylenediamine |
| 0.20 | HC Red No. 3 |
| 0.20 | HC Yellow No. 2 |
| 71.70 | dist. water |

### Preparation:

Add the components one after the other and mix.

The example can be repeated with the crosslinked polyurethanes of examples 2, 3, 4, 5, 6, 7, 8, 9, 10, 11, 12, 13 and 14. In every case, a pale brown semipermanent hair color with good properties is obtained.

### Example 61

### Shampoo

| | |
|---|---|
| 30.00 | sodium laureth sulfate |
| 6.00 | sodium cocoamphoacetate |
| 6.00 | cocamidopropylbetaine |
| 3.00 | sodium laureth sulfate, glycol distearate, |
| | cocamide MEA, Laureth-10 |
| 5.00 | polymer 1 (20% strength aqueous solution) |
| 2.00 | dimethicone |
| q.s. | perfume |
| q.s. | preservative |
| q.s. | citric acid |
| 1.00 | sodium chloride |
| ad 100 | dist. water |

### Preparation:

Weigh in and dissolve the components. Adjust pH to 6 to 7.

The example can be repeated with the crosslinked polyurethanes of examples 2, 3, 4, 5, 6, 7, 8, 9, 10, 11, 12, 13 and 14. In every case, a shampoo with good properties is obtained.

### Example 62

### Shampoo

| | |
|---|---|
| 30.00 | sodium laureth sulfate |
| 6.00 | sodium cocoamphoacetate |
| 6.00 | cocamidopropylbetaine |
| 3.00 | sodium laureth sulfate, glycol distearate, |
| | cocamide MEA, Laureth-10 |
| 5.00 | polymer 1 (20% strength aqueous solution) |
| 2.00 | amodimethicone |
| q.s. | perfume |
| q.s. | preservative |
| q.s. | citric acid |
| 1.00 | sodium chloride |
| ad 100 | dist. water |

### Preparation:

Weigh in and dissolve the components. Adjust the pH to 6 to 7.

The example can be repeated with the crosslinked polyurethanes of examples 2, 3, 4, 5, 6, 7, 8, 9, 10, 11, 12, 13 and 14. In every case, a shampoo with good properties is obtained.

### Example 63

### Shampoo

| | |
|---|---|
| 40.00 | sodium laureth sulfate |
| 10.00 | cocamidopropylbetaine |
| 3.00 | sodium laureth sulfate, glycol distearate, |
| | cocamide MEA, Laureth-10 |
| 5.00 | polymer 1 (20% strength aqueous solution) |
| 2.00 | Dow Corning 3052 |
| q.s. | perfume |
| q.s. | preservative |
| q.s. | citric acid |
| 2.00 | cocamido DEA |
| ad 100 | dist. water |

### Preparation:

Weigh in and dissolve the components. Adjust the pH to 6 to 7.

The example can be repeated with the crosslinked polyurethanes of examples 2, 3, 4, 5, 6, 7, 8, 9, 10, 11, 12, 13 and 14. In every case, a shampoo with good properties is obtained.

### Example 64

### Antidandruff Shampoo

| | |
|---|---|
| 40.00 | sodium laureth sulfate |
| 10.00 | cocamidopropylbetaine |
| 10.00 | disodium laureth sulfosuccinate |
| 2.50 | sodium laureth sulfate, glycol distearate, |
| | cocamide MEA, Laureth-10 |
| 5.00 | polymer 1 (20% strength aqueous solution) |
| 0.50 | climbazole |
| q.s. | perfume |
| q.s. | preservative |
| 0.50 | sodium chloride |
| ad 100 | dist. water |

### Preparation:

Weigh in and dissolve the components. Adjust the pH to 6 to 7.

The example can be repeated with the crosslinked polyurethanes of examples 2, 3, 4, 5, 6, 7, 8, 9, 10, 11, 12, 13 and 14. In every case, an antidandruff shampoo with good properties is obtained.

### Example 65

### Shampoo

| | |
|---|---|
| 25.00 | sodium laureth sulfate |
| 5.00 | cocamidopropylbetaine |
| 2.50 | sodium laureth sulfate, glycol distearate, |
| | cocamide MEA, Laureth-10 |
| 5.00 | polymer 1 (20% strength aqueous solution) |
| q.s. | perfume |
| q.s. | preservative |
| 2.00 | cocamido DEA |
| ad 100 | dist. water |

### Preparation:

Weigh in and dissolve the components. Adjust the pH to 6 to 7.

The example can be repeated with the crosslinked polyurethanes of examples 2, 3, 4, 5, 6, 7, 8, 9, 10, 11, 12, 13 and 14. In every case, a shampoo with good properties is obtained.

### Example 66

### Shampoo

| | |
|---|---|
| 20.00 | ammonium laureth sulfate |
| 15.00 | ammonium lauryl sulfate |
| 5.00 | cocamidopropylbetaine |
| 2.50 | sodium laureth sulfate, glycol distearate, |
| | cocamide MEA, Laureth-10 |
| 5.00 | polymer 1 (20% strength aqueous solution) |
| q.s. | perfume |
| q.s. | preservative |
| 0.50 | sodium chloride |
| ad 100 | dist. water |

### Preparation:

Weigh in and dissolve the components. Adjust the pH to 6 to 7.

The example can be repeated with the crosslinked polyurethanes of examples 2, 3, 4, 5, 6, 7, 8, 9, 10, 11, 12, 13 and 14. In every case, a shampoo with good properties is obtained.

### Example 67

### Clear Shower Gel

| | |
|---|---|
| 40.00 | sodium laureth sulfate |
| 5.00 | decyl glucoside |
| 5.00 | cocamidopropylbetaine |
| 5.00 | polymer 1 (20% strength aqueous solution) |
| 1.00 | panthenol |
| q.s. | perfume |
| q.s. | preservative |
| q.s. | citric acid |
| 2.00 | sodium chloride |
| ad 100 | dist. water |

### Preparation:

Weigh in and dissolve the components. Adjust the pH to 6 to 7.

The example can be repeated with the crosslinked polyurethanes of examples 2, 3, 4, 5, 6, 7, 8, 9, 10, 11, 12, 13 and 14. In every case, a clear shower gel with good properties is obtained.

### Example 68

### Shampoo

| | |
|---|---|
| 12.00 | sodium laureth sulfate |
| 1.50 | decyl glucoside |
| 2.50 | cocamidopropylbetaine |
| 5.00 | cocoglucoside glyceryl oleate |
| 2.00 | sodium laureth sulfate, glycol distearate, |
| | cocamide MEA, Laureth-10 |
| 5.00 | polymer 1 (20% strength aqueous solution) |
| q.s. | preservative |
| q.s. | Sunset Yellow C.I. 15 985 |
| q.s. | perfume |
| 1.00 | sodium chloride |
| ad 100 | dist. water |

### Preparation:

Weigh in and dissolve the components. Adjust the pH to 6 to 7.

The example can be repeated with the crosslinked polyurethanes of examples 2, 3, 4, 5, 6, 7, 8, 9, 10, 11, 12, 13 and 14. In every case, a shampoo with good properties is obtained.

### Example 69

### Shampoo

A

| | |
|---|---|
| 40.00 | sodium laureth sulfate |
| 5.00 | sodium C₁₂₋₁₅ Pareth-15 sulfonate |
| 5.00 | decyl glucoside |
| q.s. | perfume |
| 0.10 | phytantriol |

B

| | |
|---|---|
| ad 100 | dist. water |
| 5.00 | polymer 1 (20% strength aqueous solution) |
| 1.00 | panthenol |
| q.s. | preservative |
| 1.00 | Laureth-3 |
| q.s. | citric acid |
| 2.00 | sodium chloride |

### Preparation:

Weigh in and dissolve the components of phase A. Adjust the pH to 6 to 7. Add phase B and mix.

The example can be repeated with the crosslinked polyurethanes of examples 2, 3, 4, 5, 6, 7, 8, 9, 10, 11, 12, 13 and 14. In every case, a shampoo with good properties is obtained.

1. A crosslinked polyurethane which comprises, in incorporated form,
A) at least one polytetrahydrofuran with two terminal hydroxyl groups per molecule and a number-average molecular weight in the range from 650 to 2000,
B) at least one compound which comprises more than two active hydrogen atoms per molecule,
C) at least one compound which comprises at least two active hydrogen atoms and at least one anionogenic and/or anionic group per molecule,
D) a polyisocyanate mixture which comprises isophorone diisocyanate and hexamethylene diisocyanate, where the quantitative weight ratio of isophorone diisocyanate to hexamethylene diisocyanate is in a range from 4:1 to 15:1, and
E) at least one compound different from A) to D) with at least two active hydrogen atoms and a molecular weight of from 60 to 5000,
and the salts thereof.

2. The polyurethane according to claim 1, which additionally comprises, in incorporated form, as component F) at least one polysiloxane with at least two active hydrogen atoms per molecule.

3. The polyurethane according to claim 1, where component B) is selected from the group consisting of triols, polyols with more than three hydroxyl groups, triamines, polyamines with more than three primary and/or secondary amino groups, amine- and/or hydroxyl-group-containing polymers and mixtures thereof.

4. The polyurethane according to claim 1, where component B) comprises trimethylolpropane.

5. The polyurethane according to claim 1, where component B) comprises an amine-group- and/or hydroxyl-group-containing polymer which is obtainable by free-radical polymerization of at least one α,β-ethylenically unsaturated monomer which has at least one functional group which is selected from the group consisting of hydroxyl groups, amine groups or groups which can be converted to amine groups, and if appropriate further monomers.

6. The polyurethane according to claim 5, where component B) comprises a polymer which is selected from the group consisting of hydroxyl-group-containing polymers which have an OH number in the range from 0.3 to 60, amine-group-containing polymers which have an amine number in the range from 0.3 to 60 and hydroxyl- and amine-group-containing polymers in which the sum of hydroxyl and amine number is in the range from 0.3 to 60.

7. The polyurethane according to claim 5, where, for the polymerization, at least one monomer a) is used which is selected from the group consisting of esters of α,β-ethylenically unsaturated mono- and dicarboxylic acids with amino alcohols which have at least one primary or secondary amino group, amides of α,β-ethylenically unsaturated mono- and dicarboxylic acids with diamines which have at least one primary or secondary amino group, esters of α,β-ethylenically unsaturated mono- and dicarboxylic acids with diols, amides of α,β-ethylenically unsaturated mono- and dicarboxylic acids with amino alcohols which have a primary or secondary amino group, N,N-diallylamine, and mixtures thereof.

8. The polyurethane according to claim 7, where the monomers a) used for the polymerization comprise N-tert-butylaminoethyl methacrylate or N-tert-butylaminoethyl methacrylate is used as the sole monomer a).

9. The polyurethane according to claim 5, where component B) comprises an amine-group-containing polymer which comprises
N-tert-butylaminoethyl methacrylate
vinylimidazole
vinylpyrrolidone and
methacrylic acid
in copolymerized form.

10. The polyurethane according to claim 1, where component C) comprises dimethylolpropanoic acid.

11. The polyurethane according to claim 1, where component D) consists of a mixture of isophorone diisocyanate and hexamethylene diisocyanate.

12. The polyurethane according to claim 1, where, in component D), the quantitative weight fraction of isophorone diisocyanate is the same as or greater than the quantitative weight fraction of hexamethylene diisocyanate.

13. The polyurethane according to claim 1, where, in component D), the quantitative weight ratio of isophorone diisocyanate to hexamethylene diisocyanate is in a range from 4:1 to 10:1.

14. The polyurethane according to claim 2, comprising
12 to 35% by weight, preferably 15 to 30% by weight, of at least one compound of component A),
1 to 20% by weight, preferably 3 to 15% by weight, of at least one compound of component B) which is chosen from amine-group-containing polymers with an amine number of at least 0.1 g of KOH/g and a number-average molecular weight of at least 1000,
5 to 20% by weight, preferably 7 to 18% by weight, in particular 10 to 15% by weight, of at least one compound of component C),
23 to 60% by weight, preferably 27 to 55% by weight, in particular 32 to 50% by weight, of at least one compound of component D),
2 to 20% by weight, preferably 3 to 15% by weight, in particular 5 to 12% by weight, of at least one compound of component E),
0 to 20% by weight, preferably 0 to 10% by weight, of at least one compound of component F),
where the components add up to 100% by weight.

15. The polyurethane according to claim 2, comprising
15 to 35% by weight, preferably 18 to 30% by weight, of at least one compound of component A),
0.1 to 5% by weight, preferably 0.5 to 3% by weight, in particular 0.7 to 2.5% by weight, of at least one compound of component B), which has more than two groups reactive toward NCO groups, with a molecular weight in the range from about 80 to less than 1000 g/mol, in particular trimethylolpropane,
8 to 20% by weight, preferably 10 to 18% by weight, in particular 12 to 15% by weight, of at least one compound of component C),
25 to 60% by weight, preferably 30 to 55% by weight, in particular 35 to 50% by weight, of at least one compound of component D),
3 to 20% by weight, preferably 5 to 15% by weight, in particular 7 to 12% by weight, of at least one compound of component E),
0 to 20% by weight, preferably 0 to 18% by weight, of at least one compound of component F),
where the components add up to 100% by weight.

16. A cosmetic or pharmaceutical composition comprising
α) at least one crosslinked polyurethane according to claim 1, where, in component D), the quantitative weight ratio of isophorone diisocyanate to hexamethylene diisocyanate is in a range from 3:1 to 15.1, and
β) at least one cosmetically or pharmaceutically acceptable carrier.

17. The composition according to claim 16, where the component β) is selected from the group consisting of
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trihydric alcohols different from iii),
v) saturated acyclic and cyclic hydrocarbons,
vi) fatty acids,
vii) fatty alcohols,
viii) propellant gases,
and mixtures thereof.

18. The composition according to claim 16, further comprising at least one additive different from components α) and β) which is selected from the group consisting of cosmetically active ingredients, emulsifiers, surfactants, preservatives, perfume oils, thickeners, hair polymers, hair and skin conditioners, graft polymers, water-soluble or dispersible silicone-containing polymers, light protection agents, bleaches, gel formers, care agents, colorants, tinting agents, tanning agents, dyes, pigments, bodying agents, humectants, refatting agents, collagen, protein hydrolyzates, lipids, antioxidants, antifoams, antistats, emollients and softeners.

19. The composition according to claim 16 in the form of a gel, foam, spray, mousse, ointment, cream, emulsion, suspension, lotion, milk or paste.

20. The use of the polyurethane according to claim 1, in skin-cleansing compositions, compositions for the care and protection of the skin, nail-care compositions, preparations for decorative cosmetics or hair-treatment compositions.

21. The use according to claim 20 in hair-treatment compositions further comprising use as setting agents and/or as conditioners.

22. The use according to claim 21, where the composition is in the form of a hair gel, shampoo, setting foam, hair tonic, hairspray or hair foam.

23. The use of the polyurethane according to claim 1, as auxiliary in pharmacy, preferably as or in (a) coating(s) for solid drug forms, for modifying rheological properties, as surface-active compound, as or in (an) adhesive(s), or as or in (a) coating(s) for the textile, paper, printing and leather industry.
